(19) United States
(12) Patent Application Publication
(10) Pub. No.: US 2007/0149547 A1
(43) Pub. Date: Jun. 28, 2007

(54) Bipyridyl amides as modulators of metabotropic glutamate receptor-5

(76) Inventors: Celine Bonnefous, San Diego, CA (US); Theodore M. Kamenecka, North Palm Beach, FL (US); Jean-Michel Vernier, San Diego, CA (US)

Correspondence Address: MERCK AND CO., INC, P O BOX 2000, RAHWAY, NJ, 07065-0907, US

(21) Appl. No.: 10/589,407

(22) PCT Filed: Feb. 9, 2005
(86) PCT No.: PCT/US05/03952

§ 371 Date: Aug. 11, 2006

Related U.S. Application Data
(60) Provisional application No. 60/544,627, filed on Feb. 12, 2004.

(51) Int. Cl. A61K 31/497; A61K 31/444; C07D 403/02; C07D 401/02

(52) U.S. Cl. 514/255.05; 514/332; 544/405; 546/262

(57) ABSTRACT

The present invention is directed to novel amides such as those of Formula (I): (I) which are mGluR5 modulators useful in the treatment or prevention of diseases and conditions in which mGluR5 is involved, including but not limited to psychiatric and mood disorders such as schizophrenia, anxiety, depression, bipolar disorders, and panic, as well as in the treatment of pain, Parkinson's disease, cognitive dysfunction, epilepsy, circadian rhythm and sleep disorders, such as shift-work induced sleep disorder and jet-lag, drug addiction, drug abuse, drug withdrawal, obesity and other diseases. The invention is also directed to pharmaceutical compositions comprising these compounds. This invention further provides a method of treatment of these disorders and conditions by the administration of an effective amount of these novel amides and/or compositions containing these compounds.

### BACKGROUND OF THE INVENTION

A major excitatory neurotransmitter in the mammalian nervous system is the glutamate molecule, which binds to neurons, thereby activating cell surface receptors. Such surface receptors are characterized as either ionotropic or metabotropic glutamate receptors. The metabotropic glutamate receptors (“mGluR”) are G protein-coupled receptors that activate intracellular second messenger systems when bound to glutamate. Activation of mGluR results in a variety of cellular responses. In particular, mGluR1 and mGluR5 activate phospholipase C, which is followed by mobilizing intracellular calcium.

Modulation of metabotropic glutamate receptor subtype 5 (mGluR5) is useful in the treatment of diseases that affect the nervous system (see for example W. P. J. M Spooren et al., Trends Pharmacol. Sci., 22:331-337 (2001) and references cited therein). For example, recent evidence demonstrates the involvement of mGluR5 in nociceptive processes and that modulation of mGluR5 using mGluR5-selective compounds is useful in the treatment of various pain states, including acute, persistent and chronic pain [K Walker et al., Neuropharmacology, 40:1-9 (2001); F. Bordi, A. Ugolini Brain Res., 871:223-233 (2001)], inflammatory pain [K Walker et al., Neuropharmacology, 40:10-19 (2001); Bhave et al. Nature Neurosci. 4:417-423 (2001)] and neuropathic pain [Dogrul et al. Neurosci. Lett. 292:115-118 (2000)].

Further evidence supports the use of modulators of mGluR5 in the treatment of psychiatric and neurological disorders. For example, mGluR5-selective compounds such as 2-methyl-6-(phenylethynyl)-pyridine (“MPEP”) are effective in animal models of mood disorders, including anxiety and depression [W. P. J. M Spooren et al., J. Pharmacol. Exp. Ther., 295:1267-1275 (2000); E. Tatarczynska et al, Brit. J. Pharmacol., 132:1423-1430 (2001); A. Klodzynska et al, Pol. J. Pharmacol., 132:1423-1430 (2001)]. Gene expression data from humans indicate that modulation of mGluR5 may be useful for the treatment of schizophrenia [T. Ohnuma et al, Mol. Brain. Res., 56:207-217 (1998); ibid, Mol. Brain. Res., 85:24-31 (2000)]. Studies have also shown a role for mGluR5, and the potential utility of mGluR5-modulatory compounds, in the treatment of movement disorders such as Parkinson's disease [W. P. J. M Spooren et al., Europ. J. Pharmacol. 406:403-410 (2000); H. Awad et al., J. Neurosci. 20:7871-7879 (2000); K. Ossawa et al. Neuropharmacol. 41:413-420 (2001)]. Other research supports a role for mGluR5 modulation in the treatment of cognitive dysfunction [G. Riedel et al, Neuropharmacol. 39:1943-1951 (2000)], epilepsy [A. Chapman et al, Neuropharmacol. 39:1567-1574 (2000)] and neuroprotection [V. Bruno et al, Neuropharmacol. 39:2223-2230 (2000)]. Studies with mGluR5 knockout mice and MPEP also suggest that modulation of these receptors may be useful in the treatment of drug addiction, drug abuse and drug withdrawal [C. Chiamulera et al. Nature Neurosci. 4:873-874 (2001)].

International Patent Publications WO 01/12627 and WO 99/26927 describe heteropolycyclic compounds and their use as metabotropic glutamate receptor antagonists.

U.S. Pat. No. 3,647,809 describes pyridyl-1,2,4-oxadiazole derivatives. U.S. Pat. No. 4,022,901 describes 3-pyridyl-5-isothiocyanophenyl oxadiazoles. International Patent Publication WO 98/17652 describes oxadiazoles, WO 97/03967 describes various substituted aromatic compounds, JP 13233767A and WO 94/22846 describe various heterocyclic compounds.

Compounds that include ringed systems are described by various investigators as effective for a variety of therapies and utilities. For example, International Patent Publication No. WO 98/25883 describes ketobenzamides as calpain inhibitors, European Patent Publication No. EP 811610 and U.S. Pat. Nos. 5,679,712, 5,693,672 and 5,747,541 describe substituted benzoylguanidine sodium channel blockers, and U.S. Pat. No. 5,736,297 describes ring systems useful as a photosensitive composition.

However, there remains a need for novel compounds and compositions that therapeutically inhibit mGluR5 with minimal side effects.

### SUMMARY OF THE INVENTION

The present invention is directed to novel amides such as those of Formula (I): 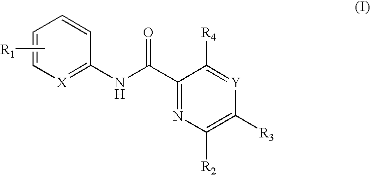
which are mGluR5 modulators useful in the treatment or prevention of diseases and conditions in which mGluR5 is involved, including but not limited to psychiatric and mood disorders such as schizophrenia, anxiety, depression, bipolar disorders, and panic, as well as in the treatment of pain, Parkinson's disease, cognitive dysfunction, epilepsy, circadian rhythm and sleep disorders, such as shift-work induced sleep disorder and jet-lag, drug addiction, drug abuse, drug withdrawal, obesity and other diseases. The invention is also directed to pharmaceutical compositions comprising these compounds. This invention further provides a method of treatment of these disorders and conditions by the administration of an effective amount of these novel amides and/or compositions containing these compounds.

### DETAILED DESCRIPTION OF THE INVENTION

In one embodiment the present invention provides novel compounds of Formula I: 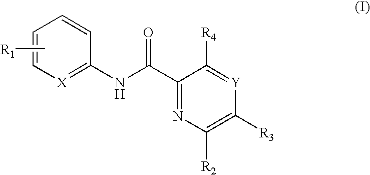
or a pharmaceutically acceptable salt thereof wherein:
X is —N—, or —C—
Y is —N—, —C—, or C-halogen.
R₁ is selected from:

1) hydrogen,

2) C₁₋₁₀alkyl,

3) C₂₋₁₀alkenyl,

4) C₂₋₁₀alkynyl

5) C₃₋₁₀cycloalkyl,

6) heterocyclyl,

7) aryl,

8) heteroaryl,

9) —NR^{d}R^{e},

10) —CO₂R^{d},

11) —OR^{d},

12) —CN, and

13) halogen,

where alkyl, alkenyl, alkynyl, cycloalkyl and heterocyclyl are optionally substituted with 1, 2, 3 or 4 substituents selected from R^{a}, and where aryl and heteroaryl are optionally substituted with 1, 2, 3, 4 or 5 substituents independently selected from R^{b};

R₂ is selected from:

1) hydrogen,

2) C₁₋₁₀alkyl,

3) C₂₋₁₀alkenyl,

4) C₂₋₁₀alkynyl,

5) C₃₋₁₀cycloalkyl,

6) heterocyclyl,

7) aryl,

8) —CN,

9) halogen,

10) —OR^{d}, and

11) heteroaryl,

where alkyl, alkenyl and alkynyl, cycloalkyl and heterocyclyl, aryl, and heteroaryl are optionally substituted with 1, 2, 3, 4 or 5 five substituents independently selected from R^{b};

R₃ is selected from:

1) aryl,

2) —NR^{d}R^{e},

3) halogen,

4) C₁₋₁₀alkyl,

5) —OR^{d},

6) hydrogen, and

7) —SR^{d},

where alkyl are optionally substituted with 1, 2, 3, 4 or 5 substituents selected from R^{a};

R² and R³ may be joined together with the atoms to which they are attached to form a saturated or unsaturated ring of 4, 5, 6 or 7 members containing 0, 1 or 2 heteroatoms independently selected from oxygen, sulfur and nitrogen;

R₄ is selected from:

1) aryl,

2) heteroaryl,

3) —NR^{d}R^{e},

4) halogen,

5) —OR^{d},

6) hydrogen, and

7) SR^{d};

where aryl and heteroaryl are optionally substituted with 1, 2, 3, 4 or 5 substituents independently selected from R^{b};

R^{a} is selected from:

1) hydrogen,

2) —OR^{d},

3) —NO₂,

4) halogen,

5) —S(O)ₘR^{d},

6) —SR^{d},

7) —S(O)ₘNR^{d}R^{e},

8) —NR^{d}R^{e},

9) —C(O)R^{d},

10) —CO₂R^{d},

11) —OC(O)R^{d},

12) —CN,

13) —SiR^{c}R^{d}R^{e},

14) —C(O)NR^{d}R^{e},

15) —NR^{d}C(O)R^{e},

16) —OC(O)NR^{d}R^{e},

17) —NR^{d}C(O)OR^{e},

18) —NR^{d}C(O)NR^{d}R^{e},

19) —CR^{d}(N—OR^{e}),

20) CF₃, and

21) —OCF₃;

R^{b} is selected from:

1) R^{a},

2) C₁₋₁₀alkyl,

3) C₂₋₁₀ alkenyl,

4) C₂₋₁₀ alkynyl,

5) C₃₋₁₀cycloalkyl,

6) heterocyclyl,

7) aryl, and

8) heteroaryl,

where alkyl, alkenyl, alkynyl, cycloalkyl, heterocyclyl, aryl, heteroaryl are optionally substituted with 1, 2, 3, 4 or 5 substituents independently selected from R^{c},

R^{c} is selected from:

1) halogen,

2) amino,

3) carboxy,

4) cyano,

5) C₁₋₄alkyl,

6) C₁₋₄alkoxy,

7) aryl,

8) aryl C₁₋₄alkyl,

9) heteroaryl,

10) hydroxy,

11) CF₃, and

12) aryloxy;

R^{d} and R^{e} are independently selected from R^{a}, C₁₋₁₀alkyl,

C₂₋₁₀alkenyl, C₂₋₁₀alkynyl and Cy, where alkyl, alkenyl, alkynyl and Cy are optionally substituted with 1, 2, 3, 4 or 5 substituents independently selected from R^{c};

or R^{d} and R^{e} together with the atoms to which they are attached form a saturated or unsaturated ring of 4, 5, 6 or 7 members containing 0, 1 or 2 heteroatoms independently selected from oxygen, sulfur and nitrogen;

Cy is independently selected from cycloalkyl, heterocyclyl, aryl, or heteroaryl; and

m is 1 or 2.

Within this embodiment is the genus of compounds wherein:

R₁ is selected from:

1) hydrogen,

2) C₁₋₆alkyl,

3) C₂₋₆alkenyl,

4) C₂₋₆alkylyl,

5) C₃₋₆cycloalkyl,

6) heterocyclyl,

7) aryl,

8) heteroaryl,

9) —NR^{d}R^{e},

10) —OR^{d},

11) —CO₂R^{d},

10) —CN,

12) halogen;

where alkyl, alkenyl, alkylyl, cycloalkyl and heterocyclyl are optionally substituted with one to four substituents selected from R^{a}, and where aryl and heteroaryl are optionally substituted with 1, 2 or 3 substituents independently selected from R^{b};

R₂ is selected from:

1) hydrogen,

2) C₁₋₆alkyl,

3) C₂₋₆alkenyl,

4) C₃₋₆cycloalkyl,

5) aryl,

6) heteroaryl,

7) —CN,

8) —OR^{d}, and

9) halogen,

where alkyl, alkenyl, cycloalkyl, aryl and heteroaryl are optionally substituted with 1, 2 or 3 substituents independently selected from R^{b};

R₃ is selected from:

1) hydrogen,

2) C₁₋₆alkyl,

3) aryl,

4) —NR^{d}R^{e},

5) —OR^{d},

6) —SR^{d},

7) halogen;

wherein alkyl is optionally substituted with 1, 2 or 3 substituents independently selected from R^{a};

R² and R³ may be joined so that together with the atoms to which R² and R³ are attached there is formed a cyclohexyl or phenyl ring;

R⁴ is selected from:

1) hydrogen,

2) aryl,

3) heteroaryl,

4) —NHR^{d},

5) —OR^{d},

6) —SR^{d},

7) halogen;

where aryl and heteroaryl are optionally substituted with 1, 2 or 3 substituents independently selected from R^{b};

R^{a} is selected from:

1) hydrogen,

2) —OR^{d},

3) halogen,

4) —NR^{d}R^{e},

5) —CN,

6) CO₂R^{d},

7) CF₃;

R^{b} is selected from:

1) R^{a},

2) C₁₋₃ alkyl,

where alkyl are optionally substituted with 1, 2 or 3 substituents independently selected from R^{c};

R^{c} is selected from:

1) hydrogen,

2) carboxy

3) C₁₋₃alkyl,

R^{d} and R^{e} are independently selected from R^{a}, C₁₋₄alkyl, cycloalkyl, aryl, or heteroaryl, where alkyl, cycloalkyl, aryl, or heteroaryl are optionally substituted with 1, 2 or 3 substituents independently selected from R^{c},

or R^{d} and R^{e} together with the atoms to which they are attached form a saturated or unsaturated ring of 4, 5, 6 or 7 members containing 0, 1 or 2 heteroatoms independently selected from oxygen, sulfur and nitrogen.

Within this genus is the sub-genus of compounds wherein:

R^{a} is selected from:

1) hydrogen,

2) —CN,

3) halogen

R^{b} is selected from R^{a}.

Within this sub-genus, is the class of compounds wherein:

R₁ is selected from:

1) hydrogen,

2) methyl, ethyl

3) —C(O)—O—CH₃,

4) pyridinyl,

5) —CN,

6) imidazolyl,

7) chloro, bromo,

8) —CH—CH, and

9) hydroxyl,

wherein alkyl and heterocyclyl are optionally substituted with 1 or 2 substituents selected from R^{a}, and where heteroaryl are optionally substituted with 1 or 2 substituents independently selected from R^{b}.

Within this sub-genus is another class of compounds wherein:

R₂ is selected from:

1) hydrogen,

2) Phenyl, optionally mono or di-substituted with a substituent selected from halo, —CH₃ and cyano,

3) CH₃, ethyl, butyl,

4) Bromo, chloro,

5) —CN,

6) —OCH₃,

7) pyridinyl, thienyl, and

8) —CF₃,

where alkyl, alkenyl, cycloalkyl, aryl and heteroaryl are optionally substituted with 1, 2 or 3 substituents independently selected from R^{b}.

Within this sub-genus is another class of compounds wherein:

R₃ is selected from:

1) hydrogen,

2) —N(CH₃)CH₃,

3) CH₃,

4) piperidinyl,

5) —S—CH₃,

6) —NCH₂CH₃,

7) —OCH₃,

8) —N—CH₂-furanyl,

9) —N—CH(CH₃)₂,

10) CF₃,

11) phenyl,

12) chloro, and

13) —NH₂,

wherein alkyl is optionally substituted with 1, 2 or 3 substituents independently selected from R^{a}.

Within this sub-genus is another class of compounds wherein:

R₂ and R₃ together with the atoms to which they are attached form a ring selected from cyclohexyl and phenyl.

Within this sub-genus is another class of compounds wherein:

R₄ is selected from:

1) hydrogen,

2) —NH₂,

3) hydroxyl,

4) —N-pyridyl,

5) —S—CH₃,

6) —N(CH₃)₂,

7) —N—C(O)—O—CH₂C═CH₂.

where aryl and heteroaryl are optionally substituted with 1, 2 or 3 substituents independently selected from R^{b}.

Within this sub-genus is another class of compounds of Formula (Ia): 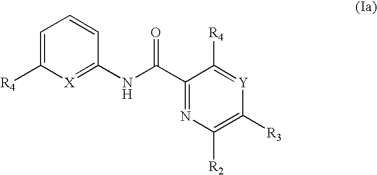
wherein
R₁ is selected from:

1) hydrogen,

2) methyl, ethyl

3) —C(O)—O—CH₃,

4) pyridinyl,

5) —CN,

6) imidazolyl,

7) chloro, bromo,

8) —CH—CH—Si(CH₃)₃,

9) —CH—CH, and

10) hydroxyl;

R₂ is selected from:

1) hydrogen,

2) Phenyl, optionally mono or di-substituted with a substituent selected from halo, —CH₃ and cyano,

3) CH₃, ethyl, butyl,

4) Bromo, chloro,

5) —CN,

6) —OCH₃,

7) pyridinyl, thienyl, and

8) —CF₃;

R₃ is selected from:

1) hydrogen,

2) —N(CH₃)CH₃,

3) CH₃,

4) piperidinyl,

5) —S—CH₃,

6) —NCH₂CH₃,

7) —OCH₃,

8) —N—CH₂-furanyl,

9) —N—CH(CH₃)₂,

10) CF₃,

11) phenyl,

12) chloro, and

13) —NH₂;

R₂ and R₃ together with the atoms to which they are attached form a ring selected from cyclohexyl and phenyl; and

R₄ is selected from:

1) hydrogen,

2) —NH₂,

3) hydroxyl,

4) —N-pyridyl,

5) —S—CH₃,

6) —N(CH₃)₂,

7) —N—C(O)—O—CH₂C═CH₂.

Within this class is a sub-class of compounds wherein:

R₃ is hydrogen or methyl.

Within this class is a sub-class of compounds wherein:

R₄ is hydroxyl, —NH₂ or —NH-aryl.

Within this class is a sub-class of compounds wherein:

R₂ is halo or methyl.

Within this class is a sub-class of compounds wherein:

R₁ is hydrogen or methyl.

Within this class is a sub-class of compounds wherein:

R₁ is hydrogen or methyl;

R₂ is halo or methyl;

R₃ is hydrogen or methyl; and

R₄ is hydroxyl, —NH₂ or —NH-aryl.

Illustrating the invention are the following compounds:
3-Amino-5,6-diphenyl-N-pyridin-2-ylpyrazine-2-carboxamide;
3-Amino-6-chloro-5-[(2-furylmethyl)amino]-N-pyridin-2-ylpyrazine-2-carboxamide;
6-Chloro-3-(dimethylamino)-5-[(2-furylmethyl)amino]-N-pyridin-2-ylpyrazine-2-carboxamide;
3-Amino-6-chloro-N-phenylpyrazine-2-carboxamide;
6-Chloro-3-(methylamino)-N-pyridin-2-ylpyrazine-2-carboxamide;
6-Methyl-N-pyridin-2-ylpyrazine-2-carboxamide;
3-Amino-6-phenyl-N-pyridin-2-ylpyrazine-2-carboxamide;
3-Amino-6-chloro-5-(methylthio)-N-pyridin-2-ylpyrazine-2-carboxamide;
6-Bromo-3-(methylthio)-N-pyridin-2-ylpyrazine-2-carboxamide;
6-Bromo-N-pyridin-2-ylpyridine-2-carboxamide;
6-Methyl-N-pyridin-2-ylpyridine-2-carboxamide;
6-Phenyl-N-pyridin-2-ylpyridine-2-carboxamide;
6-(3,5-Dichlorophenyl)-N-pyridin-2-ylpyridine-2-carboxamide;
N-Pyridin-2-yl-6-(2-thienyl)pyridine-2-carboxamide;
6-(2,4-Dimethoxyphenyl)-N-pyridin-2-ylpyridine-2-carboxamide;
N-Pyridin-2-ylquinoline-2-carboxamide;
6-Bromo-N-(6-methylpyridin-2-yl)pyridine-2-carboxamide;
6-Methyl-N-(6-methylpyridin-2-yl)pyridine-2-carboxamide;
6-Methoxy-N-pyridin-2-ylpyridine-2-carboxamide;
3-Amino-6-chloro-5-piperidin-1-yl-N-pyridin-2-ylpyrazine-2-carboxamide;
Methyl 6-{[(3-amino-6-chloropyrazin-2-yl)carbonyl]amino}pyridine-2-carboxylate;
3-Amino-6-chloro-N-(3-methylpyridin-2-yl)pyrazine-2-carboxamide;
3-amino-6-bromo-N-pyridin-2-ylpyrazine-2-carboxamide;
3-Amino-6-(2-cyanophenyl)-N-pyridin-2-ylpyrazine-2-carboxamide;
3-Amino-N-pyridin-2-yl-6-pyridin-3-ylpyrazine-2-carboxamide;
3-Amino-6-methoxy-N-(6-methylpyridin-2-yl)pyrazine-2-carboxamide;
6-Cyano-N-pyridin-2-ylpyridine-2-carboxamide;
3-Amino-6-chloro-N-(6-phenylpyridin-2-yl)pyrazine-2-carboxamide;
3-Amino-6-chloro-N-(6-cyanopyridin-2-yl)pyrazine-2-carboxamide;
3-Amino-6-chloro-N-[6-(1H-imidazol-1-yl)pyridin-2-yl]pyrazine-2-carboxamide;
3-Amino-N-2,4′-bipyridin-6-yl-6-chloropyrazine-2-carboxamide;
3-Amino-N-pyridin-2-ylpyrazine-2-carboxamide;
3-Amino-6-chloro-N-pyridin-2-ylpyrazine-2-carboxamide;
3-Amino-6-chloro-5-methoxy-N-pyridin-2-ylpyrazine-2-carboxamide;
6-Chloro-5-(dimethylamino)-N-pyridin-2-ylpyrazine-2-carboxamide;
3-Amino-6-chloro-5-(dimethylamino)-N-pyridin-2-ylpyrazine-2-carboxamide;
3-Amino-6-methyl-N-pyridin-2-ylpyrazine-2-carboxamide;
3-Amino-N-pyridin-2-yl-5-(trifluoromethyl)pyrazine-2-carboxamide;
3-Amino-N-pyridin-2-yl-6-(trifluoromethyl)pyrazine-2-carboxamide;
3-Amino-N-pyridin-2-yl-5,6,7,8-tetrahydroquinoxaline-2-carboxamide;
3-Amino-5-chloro-6-phenyl-N-pyridin-2-ylpyrazine-2-carboxamide;
3-Amino-N-pyridin-2-ylquinoxaline-2-carboxamide;
3-Amino-6-chloro-5-(ethylamino)-N-pyridin-2-ylpyrazine-2-carboxamide;
3-Amino-6-ethyl-N-pyridin-2-ylpyrazine-2-carboxamide;
6-chloro-N-pyridin-2-ylpyrazine-2-carboxamide;
3-Amino-5-(isopropylamino)-N-pyridin-2-ylpyrazine-2-carboxamide;
3-Amino-6-butyl-N-pyridin-2-ylpyrazine-2-carboxamide;
3-Amino-5,6-dimethyl-N-pyridin-2-ylpyrazine-2-carboxamide;
N-Pyridin-2-ylquinoxaline-2-carboxamide;
3-Amino-6-methyl-N-(6-methylpyridin-2-yl)pyrazine-2-carboxamide;
3-Amino-5,6-dimethyl-N-(6-methylpyridin-2-yl)pyrazine-2-carboxamide;
3-Amino-6-chloro-N-{6-[(trimethylsilyl)ethynyl]pyridin-2-yl}pyrazine-2-carboxamide;
6-Chloro-N-{6-[(trimethylsilyl)ethynyl]pyridin-2-yl}pyridine-2-carboxamide;
3-Amino-6-chloro-N-(6-ethynylpyridin-2-yl)pyrazine-2-carboxamide;
N-(6-ethynylpyridin-2-yl)-6-methylpyridine-2-carboxamide;
N-(6-ethylpyridin-2-yl)-6-methylpyridine-2-carboxamide;
N-(6-bromopyridin-2-yl)-6-methylpyridine-2-carboxamide;
6-Methyl-N-(6-phenylpyridin-2-yl)pyridine-2-carboxamide;
N-2,3′-bipyridin-6-yl-6-methylpyridine-2-carboxamide;
N-(6-cyanopyridin-2-yl)-6-methylpyridine-2-carboxamide;
N-[6-(1H-imidazol-1-yl)pyridin-2-yl]-6-methylpyridine-2-carboxamide;
3-Amino-6-cyano-N-pyridin-2-ylpyrazine-2-carboxamide;
3-Amino-6-chloro-N-(6-methylpyridin-2-yl)pyrazine-2-carboxamide;
3-Hydroxy-6-methyl-N-(6-methylpyridin-2-yl)pyridine-2-carboxamide;
Allyl(6-methyl-2-{[(6-methylpyridin-2-yl)amino]carbonyl}pyridin-3-yl)carbamate;
3-Amino-6-methyl-N-(6-methylpyridin-2-yl)pyridine-2-carboxamide;
4,6-Dichloro-N-(6-methylpyridin-2-yl)pyridine-2-carboxamide; and
6-Methyl-N-(6-methylpyridin-2-yl)-3-(pyridin-3-ylamino)pyridine-2-carboxamide.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

The term “C₀₋₁₀alkyl” includes alkyls containing 10, 9, 8, 7, 6, 5, 4, 3, 2, 1, or no carbon atoms. An alkyl with no carbon atoms, i.e., C₀, is a hydrogen atom substituent when the alkyl is a terminal group and is a direct bond when the alkyl is a bridging group.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, indenyl, fluorenyl, 1,2,3,4-tetrahydronaphalene and the like. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, indenyl, and the like. Collectively, cycloalyls and cycloalkenyls are known as “cyclyls”

The term “aryl” means an aromatic substituent which is a single ring or multiple rings fused together. When formed of multiple rings, at least one of the constituent rings is aromatic. Possible aryl substituents include phenyl and naphthyl groups.

The term “cycloalkyloxy” unless specifically stated otherwise includes a cycloalkyl group connected by a short C₁₋₂alkyl length to the oxy connecting atom.

The term “hetero” unless specifically stated otherwise includes one or more O, S, or N atoms. For example, heterocycloalkyl and heteroaryl include ring systems that contain one or more O, S, or N atoms in the ring, including mixtures of such atoms. The hetero atoms replace ring carbon atoms. Thus, for example, a heterocycloC₅alkyl is a five-member ring containing from 4 to no carbon atoms.

Examples of heteroaryls include pyridinyl, quinolinyl, isoquinolinyl, pyridazinyl, pyrimidinyl, pyrazinyl, quinoxalinyl, furyl, benzofuryl, dibenzofuryl, thienyl, benzthienyl, pyrrolyl, indolyl, pyrazolyl, indazolyl, oxazolyl, benzoxazolyl, isoxazolyl, thiazolyl, benzothiazolyl, isothiazolyl, imidazolyl, benzimidazolyl, oxadiazolyl, thiadiazolyl, triazolyl, and tetrazolyl. Examples of heterocycloalkyls include azetidinyl, pyrrolidinyl, piperidinyl, piperazinyl, morpholinyl, tetrahydrofuranyl, imidazolinyl, pyrolidin-2-one, piperidin-2-one, and thiomorpholinyl.

Similarly, the term “heteroC₀₋₄alkyl” means a heteroalkyl containing 3, 2, 1, or no carbon atoms. However, at least one heteroatom must be present. Thus, as an example, a heteroC₀₋₄alkyl having no carbon atoms but one N atom would be a —NH— if a bridging group and a —NH₂ if a terminal group. Analogous bridging or terminal groups are clear for an O or S heteroatom.

The term “amine” unless specifically stated otherwise includes primary, secondary and tertiary amines substituted with C₀₋₆alkyl.

The term “carbonyl” unless specifically stated otherwise includes a C₀₋₆alkyl substituent group when the carbonyl is terminal.

The term “halogen” includes fluorine, chlorine, bromine and iodine atoms.

The term “optionally substituted” is intended to include both substituted and unsubstituted. Thus, for example, optionally substituted aryl could represent a pentafluorophenyl or a phenyl ring. Further, optionally substituted multiple moieties such as, for example, alkylaryl are intended to mean that the aryl and the aryl groups are optionally substituted. If only one of the multiple moieties is optionally substituted then it will be specifically recited such as “an alkylaryl, the aryl optionally substituted with halogen or hydroxyl.”

Compounds described herein contain one or more double bonds and may thus give rise to cis/trans isomers as well as other conformational isomers. The present invention includes all such possible isomers as well as mixtures of such isomers.

Compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula I is shown without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. In certain embodiments of the invention said salts are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. In some embodiments the corresponding salts are citric, hydrobromic, hydrochloric, maleic, phosphoric, sulfuric, and tartaric acids.

The pharmaceutical compositions of the present invention comprise a compound represented by Formula I (or pharmaceutically acceptable salts thereof) as an active ingredient, a pharmaceutically acceptable carrier and optionally other therapeutic ingredients or adjuvants. Such additional therapeutic ingredients include, for example, i) opiate agonists or antagonists, ii) calcium channel antagonists, iii) 5HT receptor agonists or antagonists iv) sodium channel antagonists, v) NMDA receptor agonists or antagonists, vi) COX-2 selective inhibitors, vii) NK1 antagonists, viii) non-steroidal anti-inflammatory drugs (“NSAID”), ix) GABA-A receptor modulators, x) dopamine agonists or antagonists, xi) selective serotonin reuptake inhibitors (“SSR₁”) and/or selective serotonin and norepinephrine reuptake inhibitors (“SSNR₁”), xii) tricyclic antidepressant drugs, xiv) norepinephrine modulators, xv) L-DOPA, xvi) buspirone, xvii) lithium, xviii) valproate, ixx) neurontin (gabapentin), xx) olanzapine, xxi) nicotinic agonists or antagonists including nicotine, xxii) muscarinic agonists or antagonists, xxiii) heroin substituting drugs such as methadone, levo-alpha-acetylmethadol, buprenorphine and naltrexone, and xxiv) disulfiram and acamprosate. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The compounds of the present invention may be used in combination with one or more other drugs in the treatment, prevention, control, amelioration, or reduction of risk of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I may be employed. However, the combination therapy may also include therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention may be employed. Accordingly, the pharmaceutical compositions of the present invention include those that also contain one or more other active ingredients, in addition to a compound of the present invention.

Creams, ointments, jellies, solutions, or suspensions containing the compound of Formula I can be employed for topical use. Mouth washes and gargles are included within the scope of topical use for the purposes of this invention.

All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release. Oral tablets may also be formulated for immediate release, such as fast melt tablets or wafers, rapid dissolve tablets or fast dissolve films.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Dosage levels from about 0.01 mg/kg to about 140 mg/kg of body weight per day are useful in the treatment of psychiatric and mood disorders such as, for example, schizophrenia, anxiety, depression, panic, bipolar disorders, and circadian disorders, as well as being useful in the treatment of pain which are responsive to mGluR5 inhibition, or alternatively about 0.5 mg to about 7 g per patient per day. For example, schizophrenia, anxiety, depression, and panic may be effectively treated by the administration of from about 0.01 mg to 75 mg of the compound per kilogram of body weight per day, or alternatively about 0.5 mg to about 3.5 g per patient per day. Pain may be effectively treated by the administration of from about 0.01 mg to 125 mg of the compound per kilogram of body weight per day, or alternatively about 0.5 mg to about 5.5 g per patient per day. Further, it is understood that the mGluR5 inhibiting compounds of this invention can be administered at prophylactically effective dosage levels to prevent the above-recited conditions.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 1 mg to about 1000 mg of the active ingredient, typically 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In practice, the compounds represented by Formula I, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the typical oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet may contain from about 0.1 mg to about 500 mg of the active ingredient and each cachet or capsule may contain from about 0.1 mg to about 500 mg of the active ingredient. Thus, a tablet, cachet, or capsule conveniently contains 0.1 mg, 1 mg, 5 mg, 25 mg, 50 mg, 100 mg, 200 mg, 300 mg, 400 mg, or 500 mg of the active ingredient taken one or two tablets, cachets, or capsules, once, twice, or three times daily.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, may be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. The mixture may form unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

The compounds and pharmaceutical compositions of this invention have been found to exhibit biological activity as mGluR5 inhibitors. Accordingly, another aspect of the invention is the treatment in mammals of, for example, schizophrenia, anxiety (including panic, agoraphobia or other specific phobias, obsessive-compulsive disorders, post-traumatic stress disorders, acute stress disorder, generalized anxiety disorder, eating disorders, substance-induced anxiety disorders, non-specific anxiety disorders), depression, bipolar disorders, dementia, psychosis, circadian rhythm and sleep disorders, pain (including acute pain, persistent pain, chronic pain, inflammatory pain or neuropathic pain), Parkinson's disease, Alzheimer's disease, cognitive dysfunction, epilepsy, obesity, drug addiction, drug abuse and drug withdrawal (including tobacco withdrawl)—maladies that are amenable to amelioration through inhibition of mGluR5—by the administration of an effective amount of the compounds of this invention. The term “mammals” includes humans, as well as other animals such as, for example, dogs, cats, horses, pigs, and cattle. Accordingly, it is understood that the treatment of mammals other than humans is the treatment of clinical correlating afflictions to those above recited examples that are human afflictions.

Further, as described above, the compound of this invention can be utilized in combination with other therapeutic compounds. In particular, the combinations of the mGluR5 inhibiting compound of this invention can be advantageously used in combination with i) opiate agonists or antagonists, ii) calcium channel antagonists, iii) 5HT receptor agonists or antagonists iv) sodium channel antagonists, v) NMDA receptor agonists or antagonists, vi) COX-2 selective inhibitors, vii) NK1 antagonists, viii) non-steroidal anti-inflammatory drugs (“NSAID”), ix) GABA-A receptor modulators, x) dopamine agonists or antagonists, xi) selective serotonin reuptake inhibitors (“SSNRI”) and/or selective serotonin and norepinephrine reuptake inhibitors (“SSNR₁”), xii) tricyclic antidepressant drugs, xiii) norepinephrine modulators, xiv) L-DOPA, xv) buspirone, xvi) lithium, xvii) valproate, xviii) neurontin (gabapentin), xix) olanzapine, xx) nicotinic agonists or antagonists including nicotine, xxi) muscarinic agonists or antagonists, xxii) heroin substituting drugs such as methadone, levo-alpha-acetylmethadol, buprenorphine and naltrexone, and xxiii) disulfiram and acamprosate.

The weight ratio of the compound of the compound of the present invention to the other active ingredient(s) may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, or from about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s), and via the same or different routes of administration.

The subject compounds are useful in a method of modulating mGluR5 in a patient such as a mammal in need of such antagonism comprising the administration of an effective amount of the compound. The present invention is directed to the use of the compounds disclosed herein as modulators of mGluR5. In addition to primates, especially humans, a variety of other mammals can be treated according to the method of the present invention.

Another embodiment of the present invention is directed to a method for the treatment, control, amelioration, or reduction of risk of a disease or disorder in which mGluR5 is involved in a patient that comprises administering to the patient a therapeutically effective amount of a compound that is a modulator of mGluR5.

The present invention is further directed to a method for the manufacture of a medicament for modulation of mGluR5 receptors activity in humans and animals comprising combining a compound of the present invention with a pharmaceutical carrier or diluent.

The term “therapeutically effective amount” means the amount of the subject compound that will elicit the biological or medical response of a tissue, system, animal or human that is being sought by the researcher, veterinarian, medical doctor or other clinician. As used herein, the term “treatment” refers both to the treatment and to the prevention or prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The ability of the compounds of the present invention to act as mGluR5 modulators makes them useful pharmacological agents for disorders that involve mGluR5 in humans and animals, but particularly in humans.

The subject compounds are further useful in a method for the prevention, treatment, control, amelioration, or reduction of risk of the aforementioned diseases, disorders and conditions in combination with other agents.

### Assays Demonstrating Biological Activity

The compounds of this invention were tested against the hmGluR5a receptor stably expressed in mouse fibroblast Ltk⁻ cells (the hmGluR5a/L38-20 cell line) and activity was detected by changes in [Ca⁺⁺]ᵢ, measured using the fluorescent Ca⁺⁺-sensitive dye, fura-2. InsP assays were performed in mouse fibroblast Ltk⁻ cells (LM5a cell line) stably expressing hmGluR5a. The assays described in International Patent Publication WO 0116121 can be used.

Calcium Flux Assay

The activity of compounds was examined against the hmGluR5a receptor stably expressed in human embryonic kidney HEK293 cells (the hmGluR₅a cell line designated hm5a). See generally Daggett et al., Neuropharmacology 34:871-886 (1995). Receptor activity was detected by changes in intracellular calcium ([Ca²⁺]ᵢ) measured using the fluorescent calcium-sensitive dye, fura-2. The hm5a cells were plated onto 96-well plates, and loaded with 3 □M fura-2 for 1 h. Unincorporated dye was washed from the cells, and the cell plate was transferred to a 96-channel fluorimeter (SIBIA-SAIC, La Jolla, Calif.) which is integrated into a fully automated plate handling and liquid delivery system. Cells were excited at 350 and 385 nm with a xenon source combined with optical filters. Emitted light was collected from the sample through a dichroic mirror and a 510 nm interference filter and directed into a cooled CCD camera (Princeton Instruments). Image pairs were captured approximately every 1 s, and ratio images were generated after background subtraction. After a basal reading of 20 s, an EC₈₀ concentration of glutamate (10 □M) was added to the well, and the response evaluated for another 60 s. The glutamate-evoked increase in [Ca′]ᵢ in the presence of the screening compound was compared to the response of glutamate alone (the positive control).

Phosphatidylinositol Hydrolysis (PI) Assays

Inositolphosphate assays were performed as described by Berridge et al. [Berridge et al, Biochem. J. 206: 587-5950 (1982); and Nakajima et al., J. Biol. Chem. 267:2437-2442 (1992)] with slight modifications. Mouse fibroblast Ltk cells expressing hmGluR5 (hmGluR5/L38-20 cells) were seeded in 24-well plates at a density of 8×10⁵ cells/well. One □Ci of [³H]-inositol (Amersham PT6-271; Arlington Heights, Ill.; specific activity=17.7 Ci/mmol) was added to each well and incubated for 16 h at 37° C. Cells were washed twice and incubated for 45 min in 0.5 mL of standard Hepes buffered saline buffer (HBS; 125 mM NaCl, 5 mM KCl, 0.62 mM MgSO₄, 1.8 mM CaCl₂, 20 mM HEPES, 6 mM glucose, pH to 7.4). The cells were washed with HBS containing 10 mM LiCl, and 400 □L buffer added to each well. Cells were incubated at 37° C. for 20 min. For testing, 50 μL of 10× compounds used in the practice of the invention (made in BBS/LiCl (100 mM)) was added and incubated for 10 minutes. Cells were activated by the addition of 1000 M glutamate, and the plates left for 1 hour at 37° C. The incubations were terminated by the addition of 1 mL ice-cold methanol to each well. In order to isolate inositol phosphates (IPs), the cells were scraped from wells, and placed in numbered glass test tubes. One mL of chloroform was added to each tube, the tubes were mixed, and the phases separated by centrifugation. IPs were separated on Dowex anion exchange columns (AG 1-X8 100-200 mesh formate form). The upper aqueous layer (750 μL) was added to the Dowex columns, and the columns eluted with 3 mL of distilled water. The eluents were discarded, and the columns were washed with 10 mLs of 60 mM ammonium formate/5 mM Borax, which was also discarded as waste. Finally, the columns were eluted with 4 mL of 800 mM ammonium formate/0.1M formic acid, and the samples collected in scintillation vials. Scintillant was added to each vial, and the vials shaken, and counted in a scintillation counter after 2 hours. Phosphatidylinositol hydrolysis in cells treated with certain exemplary compounds was compared to phosphatidylinositol hydrolysis in cells treated with the agonist alone in the absence of compound.

In general, the compounds of this application have mGluR5 inhibitory activity as shown by IC₅₀ values of less than 10 □M in the calcium flux assay or inhibition at a concentration of 100 □M in the PI assay. The compounds should have IC₅₀ values of less than 1 □M in the calcium flux assay and IC₅₀ values of less than 10 □M in the PI assay. Alternatively, the compounds should have IC₅₀ values of less than 500 nM in the calcium flux assay and IC₅₀ values of less than 1 μM in the PI assay.

The compounds described in examples 1 to 68 have mGluR5 inhibitory activity as shown by inhibition at 100M or less in the calcium flux assay or 100 □M or less in the PI assay. Many of the compounds show inhibition at 10 □M or less in the calcium flux assay or inhibition at 100 □M or less in the PI assay. For instance, IC50 for examples 29, 38, and 58 are 0.5 □M, 1.3 □M, and 3 □M respectively.

The examples that follow are intended as an illustration of certain embodiments of the invention and no limitation of the invention is implied.

Unless specifically stated otherwise, the experimental procedures were performed under the following conditions. All operations were carried out at room or ambient temperature—that is, at a temperature in the range of 18-25° C. Evaporation of solvent was carried out using a rotary evaporator under reduced pressure (600-4000 pascals: 4.5-30 mm. Hg) with a bath temperature of up to 60° C. The course of reactions was followed by thin layer chromatography (TLC) and reaction times are given for illustration only. The structure and purity of all final products were assured by at least one of the following techniques: TLC, mass spectrometry, nuclear magnetic resonance (NMR) spectrometry or HPLC analysis. When given, yields are for illustration only. When given, NMR data is in the form of delta (□) values for major diagnostic protons, given in parts per million (ppm) relative to tetramethylsilane (TMS) as internal standard, determined at 500 MHz using the indicated solvent. Conventional abbreviations used for signal shape are: s. singlet; d. doublet; t. triplet; m. multiplet; br. broad; etc. Chemical symbols have their usual meanings; the following abbreviations are used: v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

### Methods of Synthesis

Compounds of the present invention can be prepared according to the following methods. The substituents are the same as in Formula (I) except where defined otherwise, or apparent to one in the art.

In the below-described Scheme, R₁, R₂, R₃, R₄, X, and Y are as defined above. Other variables are understood by one in the art by the context in which they are used.

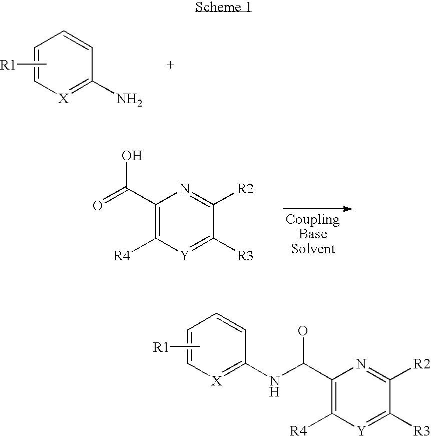

Thus, in Scheme 1, a suitably substituted amino-pyridine may be coupled with an appropriately functionalized carboxylic acid (ref.: Cragoe, E. J.; Bicking, J. B. 1968, Merck U.S. Pat. No. 3,361,748) in the presence of a common peptide coupling reagent (such as DCC, N,N′-carbonyldiimidazole, HATU, PyOAP, etc. . . . .) (For a review of peptide formation using coupling reagents, see Klausner, Y. S.; Bodansky, M. Synthesis, 1972, 453-463). Typically a base (e.g. K₂CO₃, Cs₂CO₃, K₃PO₄, Et₃N, NaOtBu, KOtBu, etc. . . . ) will also be present and the reaction carried out in a suitable solvent (DCM, THF, DME, DMF, DMAC, CH₃CN, dioxane, toluene, benzene, etc. . . . ). The reaction is conducted under an inert atmosphere (N₂ or argon) at room temperature but could be done at a temperature between 20-100° C. The reaction mixture is then maintained at a suitable temperature for a time in the range of about 2 up to 48 h with 12 h typically being sufficient. The product from the reaction can be isolated and purified employing standard techniques, such as solvent extraction, chromatography, crystallization, distillation and the like.

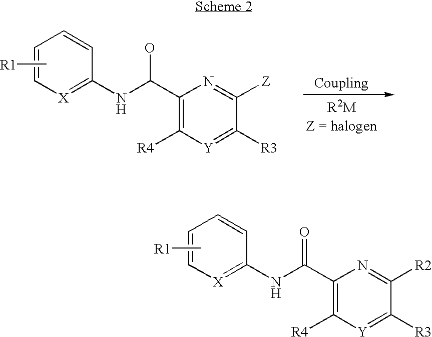

Another embodiment of the present invention is illustrated in Scheme 2 where Z is a halogen atom (Cl, Br, I). This amide product may then be coupled with an R²-group under metal-catalyzed cross-coupling conditions where M is a metallic or metalloid species such as B(OR)₂, LiR, RMgHal, SnR₃, RZnHal, SiR₃, NaR and the like which is capable of undergoing a metal-catalyzed cross-coupling reaction. The coupling may be promoted by a homogeneous catalyst such as Pd(Ph₃P)₄, PdCl₂ (Ph₃P)₂, Pd₂ dba₃, Pd(OAc)₂, PdCl₂dppf, CuI and the like. Typically a base (e.g. K₂CO₃, Cs₂CO₃, K₃PO₄, Et₃N, NaOtBu, KOtBu, etc. . . . ) will also be present and the reaction carried out in a suitable solvent (DCM, THF, MeOH, DME, DMF, DMAC, CH₃CN, dioxane, toluene, benzene, etc. . . . ). Additionally, ligands such as BINAP, di-tert-butyl phosphinobiphenyl, di-cyclohexylphosphino biphenyl, tri tert-butylphosphine, XANTPHOS, triphenylarsine, trans-1,2-cyclohexanediamine, 1,10-phenanthroline and the like may be added. Other promoters may also be used such as CsF, etc. . . . The reaction mixture is maintained at rt, or heated to a temperature between 30° C. to 150° C. The reaction mixture is then maintained at a suitable temperature for a time in the range of about 4 up to 48 h, with about 18 h typically being sufficient (for Pd examples, see Miyaura, N.; Suzuki, A. Chem. Rev. 1995, 95, 2457-2483. For Cu examples, see Klaspar, A.; Antilla, J.; Huang, X.; Buchwald, S. J. Am. Chem. Soc. 2001, 123, 7723-7729). Alternatively, the reaction may be carried out under microwave irradiation in a sealed tube. These reactions are typically conducted at a temperature between 110-180° C. for a time range of 5 min to 2 h with 20 min typically being sufficient. The product from the reaction can be isolated and purified employing standard techniques, such as solvent extraction, chromatography, crystallization, distillation and the like.

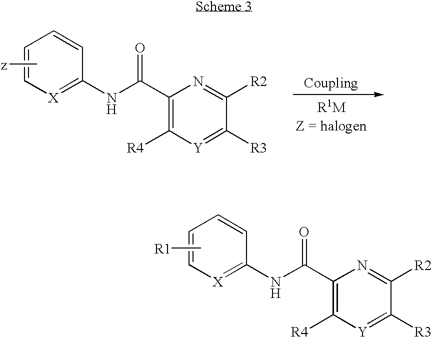

Another embodiment of the present invention is illustrated in Scheme 3 where Z is a halogen atom (Cl, Br, I). This amide product may then be coupled with an R₁-group under metal-catalyzed cross-coupling conditions where M is a metallic or metalloid species such as B(OR)₂, LiR, RMgHal, SnR₃, RZnHal, SiR₃, NaR and the like which is capable of undergoing a metal-catalyzed cross-coupling reaction. The coupling may be promoted by a homogeneous catalyst such as Pd(Ph₃P)₄, PdCl₂ (Ph₃P)₂, Pd₂ dba₃, Pd(OAc)₂, PdCl₂dppf, CuI and the like. Typically a base (e.g. K₂CO₃, Cs₂CO₃, K₃PO₄, Et₃N, NaOtBu, KOtBu, etc. . . . ) will also be present and the reaction carried out in a suitable solvent (DCM, THF, DME, DMF, DMAC, MeOH, CH₃CN, dioxane, toluene, benzene, etc. . . . ). Additionally, ligands such as BINAP, di-tert-butyl phosphinobiphenyl, di-cyclohexylphosphino biphenyl, tri tert-butylphosphine, XANTPHOS, triphenylarsine and the like, trans-1,2-cyclohexanediamine, 1,10-phenanthroline may be added. Other promoters may also be used such as CsF etc. . . . The reaction mixture is maintained at rt, or heated to a temperature between 30° C. to 150° C. The reaction mixture is then maintained at a suitable temperature for a time in the range of about 4 up to 48 h, with about 18 h typically being sufficient (for Pd examples, see Miyaura, N.; Suzuki, A. Chem. Rev. 1995, 95, 2457-2483. For Cu examples, see Klaspar, A.; Antilla, J.; Huang, X.; Buchwald, S. J. Am. Chem. Soc. 2001, 123, 7723-7729). Alternatively, the reaction may be carried out under microwave irradiation in a sealed tube. These reactions are typically conducted at a temperature between 110-180° C. for a time range of 5 min to 2 h with 20 min typically being sufficient. The product from the reaction can be isolated and purified employing standard techniques, such as solvent extraction, chromatography, crystallization, distillation and the like.

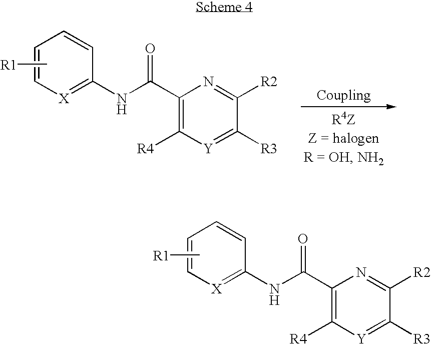

Another embodiment of the present invention is illustrated in Scheme 4 where Z is a halogen atom (Cl, Br, I). This amide product may then be coupled with an R⁴-group under metal-catalyzed cross-coupling conditions. The coupling may be promoted by a homogeneous catalyst such as Pd(Ph₃P)₄, PdCl₂ (Ph₃P)₂, Pd₂ dba₃, Pd(OAc)₂, PdCl₂dppf, CuI and the like. Typically a base (e.g. K₂CO₃, Cs₂CO₃, K₃PO₄, Et₃N, NaOtBu, KOtBu, etc. . . . ) will also be present and the reaction carried out in a suitable solvent (DCM, THF, MeOH, DME, DMF, DMAC, CH₃CN, dioxane, toluene, benzene, etc. . . . ). Additionally, ligands such as BINAP, di-tert-butyl phosphinobiphenyl, di-cyclohexylphosphino biphenyl, tri tert-butylphosphine, XANTPHOS, triphenylarsine, trans-1,2-cyclohexanediamine, 1,10-phenanthroline and the like may be added. Other promoters may also be used such as CsF, etc. . . . The reaction mixture is maintained at rt, or heated to a temperature between 30° C. to 150° C. The reaction mixture is then maintained at a suitable temperature for a time in the range of about 4 up to 48 h, with about 18 h typically being sufficient (for examples see Yang, B. H.; Buchwald, S. L. J. Organometallic Chem. 1999, 576, 125-146). Alternatively, the reaction may be carried out under microwave irradiation in a sealed tube. These reactions are typically conducted at a temperature between 110-180° C. for a time range of 5 min to 2 h with 20 min typically being sufficient. The product from the reaction can be isolated and purified employing standard techniques, such as solvent extraction, chromatography, crystallization, distillation and the like.

Exemplifying the invention is the use of the compounds disclosed in the Examples and herein. Specific compounds within the present invention include a compound which selected from the group consisting of the compounds disclosed in the following Examples and pharmaceutically acceptable salts thereof and individual diastereomers thereof.

### EXAMPLE 1

### 3-Amino-5,6-diphenyl-N-pyridin-2-ylpyrazine-2-carboxamide

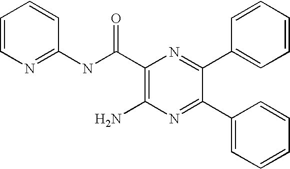 General Procedure A: Amide Formation Starting from an Acid.

A mixture of pyridin-2-amine (48 mg, 0.51 mmol), 3-amino-5,6-diphenylpyrazine-2-carboxylic acid (100 mg, 0.34 mmol), N-[(dimethylamino)(3H-[1,2,3]triazolo[4,5-b]pyridin-3-yloxy)methylene]-N-methylmethanaminium hexafluorophosphate (155 mg, 0.41 mmol), and ethyl(diisopropyl)amine (0.18 mL, 1.02 mmol) in CH₂Cl₂ (5 mL) was stirred at room temperature for 24 h. A mixture of CH₂Cl₂ and MeOH was added to the reaction mixture to dissolve the insolubles. Purification by flash chromatography on silica gel eluting with a mixture of EtOAc/Hexane afforded 107 mg of desired compound as a yellow solid. ¹H NMR (CDCl₃, 500 MHz) □ 10.34 (s, 1H), 8.38 (m, 2H), 7.77 (t, 1H), 7.44 (d, 1H), 7.39 (m, 2H), 7.35 (m, 1H), 7.28 (m, 6H), 7.08 (t, 1H). MS (ESI⁺) 368 (M⁺+1).

### EXAMPLE 2

### 3-Amino-6-chloro-5-[(2-furylmethyl)amino]-N-pyridin-2-ylpyrazine-2-carboxamide

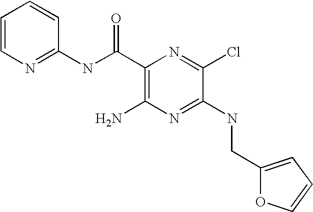

3-Amino-6-chloro-5-[(2-furylmethyl)amino]-N-pyridin-2-ylpyrazine-2-carboxamide was synthesized according to general procedure A using pyridin-2-amine and 3-amino-6-chloro-5-[(2-furylmethyl)amino]pyrazine-2-carboxylic acid and pyridin-2-amine as starting materials. ¹H NMR (CDCl₃, 500 MHz) □ 9.73 (s, 1H), 8.33 (d, 1H), 8.32 (d, 1H), 7.71 (t, 1H), 7.40 (s, 1H), 7.02 (t, 1H), 6.35 (d, 1H), 6.30 (d, 1H), 5.75 (s, 1H), 4.65 (s, 2H), 1.62 (s, 2H). MS (ESI⁺) 345 (M⁺+1).

### EXAMPLE 3

### 6-Chloro-3-(dimethylamino)-5-[(2-furylmethyl)amino]1-N-pyridin-2-ylpyrazine-2-carboxamide

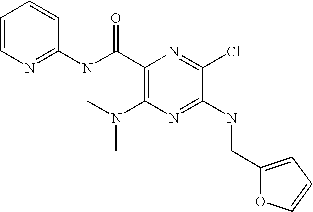

6-Chloro-3-(dimethylamino)-5-[(2-furylmethyl)amino]-N-pyridin-2-ylpyrazine-2-carboxamide was synthesized according to general procedure A using pyridin-2-amine and 6-chloro-3-(dimethylamino)-5-[(2-furylmethyl)amino]pyrazine-2-carboxylic acid as starting materials. ¹H NMR (CDCl₃, 500 MHz) □ 9.75 (s, 1H), 8.63 (s, 1H), 8.32 (d, 1H), 8.26 (d, 1H), 7.69 (1, 1H), 7.35 (s, 1H), 7.02 (t, 1H), 6.31 (s, 1H), 6.22 (s, 1H), 4.65 (s, 2H), 3.23 (s, 6H). MS (ESI⁺) 373 (M⁺+1).

### EXAMPLE 4

### 3-Amino-6-chloro-N-phenylpyrazine-2-carboxamide

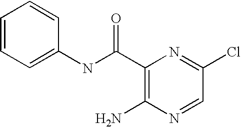

3-Amino-6-chloro-N-phenylpyrazine-2-carboxamide was synthesized according to general procedure A using aniline and 3-amino-6-chloropyrazine-2-carboxylic acid as starting materials. ¹H NMR (CDCl₃, 500 MHz) □ 9.46 (s, 1H), 8.35 (s, 1H), 7.70 (d, 2H), 7.34 (t, 2H), 7.17 (t, 1H), 1.71 (s, 2H). MS (ESI⁺) 249 (M⁺+1).

### EXAMPLE 5

### 6-Chloro-3-(methylamino)-N-pyridin-2-ylpyrazine-2-carboxamide

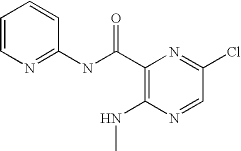 General Procedure B: Amide Formation Starting from an Ester.

An aqueous LiOH solution (1M, 1 mL) was added to a cooled solution of methyl 6-chloro-3-(methylamino)pyrazine-2-carboxylate (44 mg, 0.22 mmol) was dissolved in CH₂Cl₂ (2 μL) and the resulting mixture was stirred for 2 h. The pH was adjusted to 6 by adding an aqueous 1M HCl solution. The aqueous layer was then extracted with EtOAc (3×). The organics were combined, dried over Na₂SO₄, and evaporated to dryness to yield 6-chloro-3-(methylamino)pyrazine-2-carboxylic acid as a yellow solid.

6-Chloro-3-(methylamino)-N-pyridin-2-ylpyrazine-2-carboxamide was synthesized according to general procedure A using pyridin-2-amine and 6-chloro-3-(methylamino)pyrazine-2-carboxylic acid as starting materials. ¹H NMR (CDCl₃, 500 MHz) □ 10.06 (s, 1H), 8.48 (s, 1H), 8.36 (d, 1H), 8.25 (m, 2H), 7.73 (t, 1H), 7.09 (t, 1H), 3.08 (s, 3H). MS (ESI⁺) 264 (M⁺+1).

### EXAMPLE 6

### 6-Methyl-N-pyridin-2-ylpyrazine-2-carboxamide

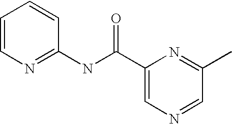

6-Methyl-N-pyridin-2-ylpyrazine-2-carboxamide was synthesized according to general procedure A using pyridin-2-amine and 6-methylpyrazine-2-carboxylic acid as starting materials. ¹H NMR (CDCl₃, 500 MHz) □ 10.26 (s, 1H), 9.31 (s, 1H), 8.64 (s, 1H), 8.41 (m, 2H), 7.79 (t, 1H), 7.13 (t, 1H), 2.66 (s, 3H). MS (ESI⁺) 215 (M⁺+1).

### EXAMPLE 7

### 3-Amino-6-phenyl-N-pyridin-2-ylpyrazine-2-carboxamide

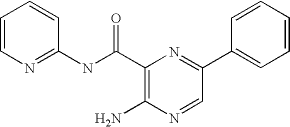

3-Amino-6-phenyl-N-pyridin-2-ylpyrazine-2-carboxamide was synthesized according to general procedure B using pyridin-2-amine and methyl 3-amino-6-phenylpyrazine-2-carboxylate as starting materials. ¹H NMR (CDCl₃, 500 MHz) □ 10.39 (s, 1H), 8.78 (s, 1H), 8.39 (m, 2H), 7.96 (d, 2H), 7.78 (t, 1H), 7.50 (t, 2H), 7.43 (d, 1H), 7.10 (t, 1H), 1.62 (s, 2H). MS (ESI⁺) 292 (M⁺+1).

### EXAMPLE 8

### 3-Amino-6-chloro-5-(methylthio)-N-pyridin-2-ylpyrazine-2-carboxamide

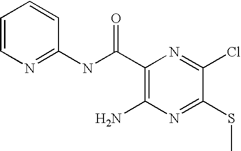

3-Amino-6-chloro-5-(methylthio)-N-pyridin-2-ylpyrazine-2-carboxamide was synthesized according to general procedure B using pyridin-2-amine and methyl 3-amino-6-chloro-5-(methylthio)pyrazine-2-carboxylate as starting materials. ¹H NMR (CDCl₃, 500 MHz) □ 9.91 (s, 1H), 8.35 (d, 1H), 8.28 (d, 1H), 7.75 (t, 1H), 7.07 (t, 1H), 2.58 (s, 3H). MS (ESI⁺) 296 (M⁺+1).

### EXAMPLE 9

### 6-Bromo-3-(methylthio)-N-pyridin-2-ylpyrazine-2-carboxamide

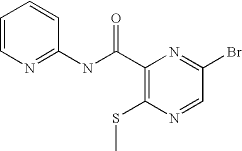

6-Bromo-3-(methylthio)-N-pyridin-2-ylpyrazine-2-carboxamide was synthesized according to general procedure B using pyridin-2-amine and methyl 6-bromo-3-(methylthio)pyrazine-2-carboxylate as starting materials. ¹H NMR (CDCl₃, 500 MHz) □ 10.05 (s, 1H), 8.66 (s, 1H), 8.45 (d, 1H), 8.36 (d, 1H), 7.75 (t, 1H), 7.11 (t, 1H), 2.55 (s, 3H). MS (ESI⁺) 325 (M⁺).

### EXAMPLE 10

### 6-Bromo-N-pyridin-2-ylpyridine-2-carboxamide

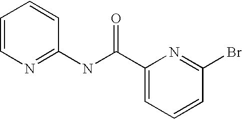

6-Bromo-N-pyridin-2-ylpyridine-2-carboxamide was synthesized according to general procedure A using pyridin-2-amine and 6-bromopyridine-2-carboxylic acid as starting materials. ¹H NMR (CDCl₃, 500 MHz) □ 10.22 (s, 1H), 8.39 (m, 2H), 8.25 (d, 1H), 7.79 (m, 2H), 7.67 (d, 1H), 7.11 (t, 1H). MS (ESI⁺) 278 (M⁺).

### EXAMPLE 11

### 6-Methyl-N-pyridin-2-ylpyridine-2-carboxamide

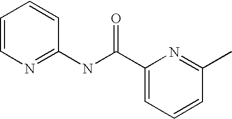

6-Methyl-N-pyridin-2-ylpyridine-2-carboxamide was synthesized according to general procedure A using pyridin-2-amine and 6-methylpyridine-2-carboxylic acid as starting materials. ¹H NMR (CDCl₃, 500 MHz) □ 10.60 (s, 1H), 8.45 (d, 1H), 8.37 (d, 1H), 8.10 (d, 1H), 7.76 (m, 2H), 7.33 (d, 1H), 7.07 (t, 1H), 2.61 (s, 3H). MS (ESI⁺) 214 (M⁺+1).

### EXAMPLE 12

### 6-Phenyl-N-pyridin-2-ylpyridine-2-carboxamide

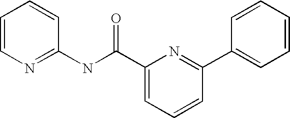 General Procedure C: Microwave Assisted Suzuki Coupling.

A mixture of 6-bromo-N-pyridin-2-ylpyridine-2-carboxamide (50 mg, 0.18 mmol), phenylboronic acid (26 mg, 0.22 mmol), PdCl₂(PPh₃)₂ (6.5 mg, 0.01 mmol), and potassium carbonate (50 mg, 0.36 mmol) in DME/H₂O (1:1, 3 mL) was stirred at 160 C in a microwave for 10 min. The resulting black mixture was cooled to room temperature, filtered through celite, and poured into a EtOAc/brine mixture. The two layers were separated and the aqueous was extracted with EtOAc (3×). The organics were combined, dried over sodium sulfate, filtered, and evaporated to dryness. The residue was purified by flash chromatography on silica gel eluting with a mixture of EtOAc/Hexane to yield 38 mg of 6-phenyl-N-pyridin-2-ylpyridine-2-carboxamide. ¹H NMR (CDCl₃, 500 MHz) □ 10.59 (s, 1H), 8.47 (d, 1H), 8.39 (d, 1H), 8.25 (d, 1H), 8.05 (d, 2H), 7.94 (m, 2H), 7.75 (t, 1H), 7.52 (m, 3H), 7.10 (t, 1H). MS (ESI⁺) 276 (M⁺+1).

### EXAMPLE 13

### 6-(3,5-Dichlorophenyl)-N-pyridin-2-ylpyridine-2-carboxamide

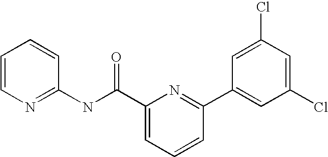

6-(3,5-Dichlorophenyl)-N-pyridin-2-ylpyridine-2-carboxamide was synthesized according to general procedure C using 6-bromo-N-pyridin-2-ylpyridine-2-carboxamide and (3,5-dichlorophenyl)boronic acid as starting materials. ¹H NMR (CDCl₃, 500 MHz) □ 10.41 (s, 1H), 8.45 (d, 1H), 8.41 (d, 1H), 8.34 (d, 1H), 8.03 (t, 1H), 7.93 (s, 2H), 7.89 (d, 1H), 7.79 (t, 1H), 7.47 (s, 1H), 7.12 (t, 1H). MS (ESI⁺) 344 (M⁺+1).

### EXAMPLE 14

### N-Pyridin-2-yl-6-(2-thienyl)pyridine-2-carboxamide

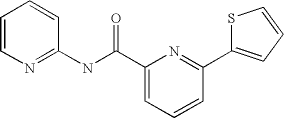

N-Pyridin-2-yl-6-(2-thienyl)pyridine-2-carboxamide was synthesized according to general procedure C using 6-bromo-N-pyridin-2-ylpyridine-2-carboxamide and 2-thienylboronic acid as starting materials. ¹H NMR (CDCl₃, 500 MHz) □ 10.46 (s, 1H), 8.44 (d, 1H), 8.42 (d, 1H), 8.15 (d, 1H), 7.91 (t, 1H), 7.83 (d, 1H), 7.75 (t, 1H), 7.71 (d, 1H), 7.45 (d, 1H), 7.16 (d, 1H), 7.10 (t, 1H). MS (ESI⁺) 282 (M⁺+1).

### EXAMPLE 15

### 6-(2,4-Dimethoxyphenyl)-N-pyridin-2-ylpyridine-2-carboxamide

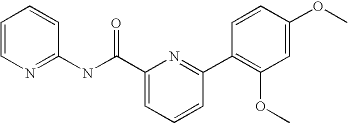

6-(2,4-Dimethoxyphenyl)-N-pyridin-2-ylpyridine-2-carboxamide was synthesized according to general procedure C using 6-bromo-N-pyridin-2-ylpyridine-2-carboxamide and (2,4-dimethoxyphenyl)boronic acid as starting materials. ¹H NMR (CDCl₃, 500 MHz) □ 10.60 (s, 1H), 8.47 (d, 1H), 8.37 (d, 1H), 8.17 (d, 1H), 8.08 (d, 1H), 7.95 (d, 1H), 7.88 (t, 1H), 7.75 (t, 1H), 7.07 (t, 1H), 6.68 (d, 1H), 6.59 (s, 1H), 3.89 (s, 6H). MS (ESI⁺) 336 (M⁺+1).

### EXAMPLE 16

### N-Pyridin-2-ylquinoline-2-carboxamide

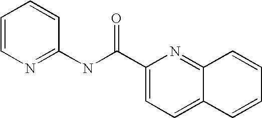

N-Pyridin-2-ylquinoline-2-carboxamide was synthesized according to general procedure A using pyridin-2-amine and quinoline-2-carboxylic acid as starting materials. ¹H NMR (CDCl₃, 500 MHz) □10.76 (s, 1H), 8.49 (d, 1H), 8.42 (d, 1H), 8.36 (m, 2H), 8.18 (d, 1H), 7.89 (d, 1H), 7.78 (m, 2H), 7.64 (t, 1H), 7.10 (t, 1H). MS (ESI⁺) 250 (M⁺+1).

### EXAMPLE 17

### 6-Bromo-N-(6-methylpyridin-2-yl)pyridine-2-carboxamide

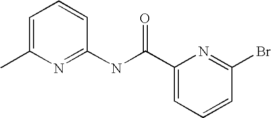

6-Bromo-N-(6-methylpyridin-2-yl)pyridine-2-carboxamide was synthesized according to general procedure A using 6-methylpyridin-2-amine and 6-bromopyridine-2-carboxylic acid as starting materials. ¹H NMR (CDCl₃, 500 MHz) □ 10.09 (s, 1H), 8.25 (d, 1H), 8.20 (d, 1H), 7.76 (t, 1H), 7.66 (m, 2H), 6.96 (d, 1H), 2.52 (s, 3H). MS (ESI⁺) 292 (M⁺).

### EXAMPLE 18

### 6-Methyl-N-(6-methylpyridin-2-yl)pyridine-2-carboxamide

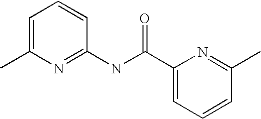

6-Methyl-N-(6-methylpyridin-2-yl)pyridine-2-carboxamide was synthesized according to general procedure A using 6-methylpyridin-2-amine and 6-methylpyridine-2-carboxylic acid as starting materials. ¹H NMR (CDCl₃, 500 MHz) □ 10.47 (s, 1H), 8.25 (d, 1H), 8.08 (d, 1H), 7.75 (t, 1H), 7.62 (t, 1H), 7.31 (d, 1H), 6.94 (d, 1H), 2.64 (s, 3H), 2.52 (s, 3H). MS (ESI⁺) 228 (M⁺+1).

### EXAMPLE 19

### 6-Methoxy-N-pyridin-2-ylpyridine-2-carboxamide

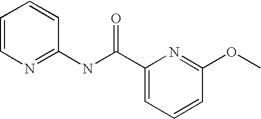

6-Methoxy-N-pyridin-2-ylpyridine-2-carboxamide was synthesized according to general procedure B using pyridin-2-amine and methyl 6-methoxypyridine-2-carboxylate as starting materials. ¹H NMR (CDCl₃, 500 MHz) □ 10.20 (s, 1H), 8.44 (d, 1H), 8.36 (d, 1H), 7.90 (d, 1H), 7.77 (m, 2H), 7.08 (t, 1H), 6.96 (d, 1H), 4.07 (s, 1H). MS (ESI⁺) 230 (M⁺+1).

### EXAMPLE 20

### 3-Amino-6-chloro-5-piperidin-1-yl-N-pyridin-2-ylpyrazine-2-carboxamide

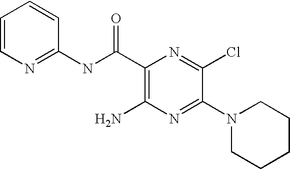

3-Amino-6-chloro-5-piperidin-1-yl-N-pyridin-2-ylpyrazine-2-carboxamide was synthesized according to general procedure B using pyridin-2-amine and methyl 3-amino-6-chloro-5-piperidin-1-ylpyrazine-2-carboxylate as starting materials. ¹H NMR (CDCl₃, 500 MHz) □ 9.84 (s, 1H), 8.34 (d, 1H), 8.28 (d, 1H), 7.71 (t, 1H), 7.02 (t, 1H), 3.57 (m, 4H), 1.68 (m, 6H). MS (ESI⁺) 333 (M⁺+1).

### EXAMPLE 21

### Methyl 6-{[(3-amino-6-chloropyrazin-2-yl)carbonyl]amino}pyridine-2-carboxylate

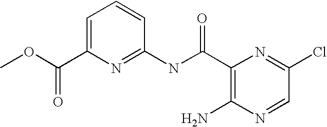

Methyl 6-{[(3-amino-6-chloropyrazin-2-yl)carbonyl]amino}pyridine-2-carboxylate was synthesized according to general procedure A using methyl 6-aminopyridine-2-carboxylate and 3-amino-6-chloropyrazine-2-carboxylic acid as starting materials. ¹H NMR (CDCl₃, 500 MHz) □ 9.94 (s, 1H), 8.21 (s, 1H), 8.09 (d, 1H), 7.64 (t, 1H), 6.95 (d, 1H), 2.51 (s, 3H). MS (ESI⁺) 307 (M⁺).

### EXAMPLE 22

### 3-Amino-6-chloro-N-(3-methylpyridin-2-yl)pyrazine-2-carboxamide

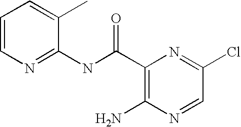

3-Amino-6-chloro-N-(3-methylpyridin-2-yl)pyrazine-2-carboxamide was synthesized according to general procedure A using 3-methylpyridin-2-amine and 3-amino-6-chloropyrazine-2-carboxylic acid as starting materials. ¹H NMR (CDCl₃, 500 MHz) □ 9.73 (s, 1H), 8.37 (d, 1H), 8.23 (s, 1H), 7.61 (d, 1H), 7.14 (t, 1H), 2.36 (s, 3H). MS (ESI⁺) 264 (M⁺+1).

### EXAMPLE 23

### 3-amino-6-bromo-N-pyridin-2-ylpyrazine-2-carboxamide

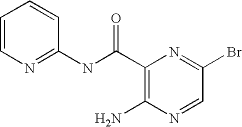

3-amino-6-bromo-N-pyridin-2-ylpyrazine-2-carboxamide was synthesized according to general procedure A using pyridin-2-amine and 3-amino-6-bromopyrazine-2-carboxylic acid as starting materials. MS (ESI⁺) 293.9 (M⁺).

### EXAMPLE 24

### 3-Amino-6-(2-cyanophenyl)-N-pyridin-2-ylpyrazine-2-carboxamide

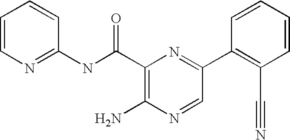

A mixture of 3-amino-6-bromo-N-pyridin-2-ylpyrazine-2-carboxamide (100 mg, 0.34 mmol), bromo(2-cyanophenyl)zinc (1 M in THF, 1 mL, 0.51 mmol), and Pd(PPh₃)₄ (39 mg, 0.034 mmol) in THF (2 mL) was heated in the microwave to 160° C. for 25 min. The resulting black mixture was cooled to room temperature, filtered through celite, and poured into a EtOAc/brine mixture. The two layers were separated and the aqueous was extracted with EtOAc (3×). The organics were combined, dried over sodium sulfate, filtered, and evaporated to dryness. The residue was purified by flash chromatography on silica gel eluting with a mixture of EtOAc/Hexane to yield 9 mg of 3-amino-6-(2-cyanophenyl)-N-pyridin-2-ylpyrazine-2-carboxamide as a yellow solid. ¹H NMR (CDCl₃, 500 MHz) □ 10.46 (s, 1H), 8.68 (s, 1H), 8.42 (d, 1H), 8.30 (d, 1H), 7.86-7.69 (m, 4H), 7.51 (t, 1H), 7.08 (t, 1H), 1.61 (s, 2H). MS (ESI⁺) 317 (M⁺+1).

### EXAMPLE 25

### 3-Amino-N-pyridin-2-yl-6-pyridin-3-ylpyrazine-2-carboxamide

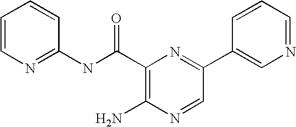

3-Amino-N-pyridin-2-yl-6-pyridin-3-ylpyrazine-2-carboxamide was synthesized according to general procedure C using 3-amino-6-bromo-N-pyridin-2-ylpyrazine-2-carboxamide and 3-(diethylboryl)pyridine as starting materials. ¹H NMR (CDCl₃, 500 MHz) □ 10.31 (s, 1H), 9.18 (s, 1H), 8.86 (s, 1H), 8.66 (d, 1H), 8.39 (d, 1H), 8.36 (d, 1H), 8.28 (d, 1H), 7.78 (t, 1H), 7.44 (t, 1H), 7.11 (t, 1H). MS (ESI⁺) 293 (M⁺+1).

### EXAMPLE 26

### 3-Amino-6-methoxy-N-(6-methylpyridin-2-yl)pyrazine-2-carboxamide

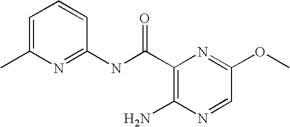

3-Amino-6-bromo-N-(6-methylpyridin-2-yl)pyrazine-2-carboxamide was synthesized according to general procedure B using 6-methylpyridin-2-amine and methyl 3-amino-6-bromopyrazine-2-carboxylate as starting materials.

A mixture of 3-amino-6-bromo-N-(6-methylpyridin-2-yl)pyrazine-2-carboxamide (100 mg, 0.32 mmol), copper iodide (6 mg, 0.032 mmol), 1,10-phenanthroline (12 mg, 0.064 mmol), and cesium carbonate (208 mg, 0.64 mmol) in MeOH (2 mL) was heated in a microwave at 140° C. for 5 min. The resulting mixture was cooled to room temperature, filtered through celite, and poured into a EtOAc/brine mixture. The two layers were separated and the aqueous was extracted with EtOAc (3×). The organics were combined, dried over sodium sulfate, filtered, and evaporated to dryness. The residue was purified by flash chromatography on silica gel eluting with a mixture of EtOAc/Hexane to yield 9 mg of 3-amino-6-methoxy-N-(6-methylpyridin-2-yl)pyrazine-2-carboxamide. ¹H NMR (CDCl₃, 500 MHz) □ 9.87 (s, 1H), 8.20 (d, 1H), 8.04 (s, 1H), 7.65 (t, 1H), 6.34 (s, 2H), 4.02 (s, 3H), 2.50 (s, 3H). MS (ESI⁺) 260 (M⁺+1).

### EXAMPLE 27

### 6-Cyano-N-pyridin-2-ylpyridine-2-carboxamide

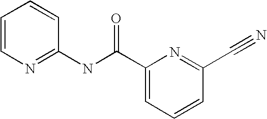 General Procedure D: Palladium Catalyzed Cyanation.

A mixture of 6-bromo-N-pyridin-2-ylpyridine-2-carboxamide (100 mg, 0.36 mmol), sodium cyanide (27 mg, 0.54 mmol), Pd(PPh₃)₄ (21 mg, 0.018 mmol), and copper iodide (7 mg, 0.036 mmol) in acetonitrile (2 mL) was heated in the microwave at 180° C. for 10 min. The resulting mixture was cooled to room temperature, filtered through celite, and poured into a EtOAc/brine mixture. The two layers were separated and the aqueous was extracted with EtOAc (3×). The organics were combined, dried over sodium sulfate, filtered, and evaporated to dryness. The residue was purified by flash chromatography on silica gel eluting with a mixture of EtOAc/Hexane to yield 23 mg of 6-cyano-N-pyridin-2-ylpyridine-2-carboxamide. ¹H NMR (CDCl₃, 500 MHz) □ 10.22 (s, 1H), 8.55 (d, 1H), 8.40 (m, 2H), 8.12 (t, 1H), 7.90 (d, 1H), 7.79 (t, 1H), 7.13 (t, 1H). MS (ESI⁺) 225 (M⁺+1).

### EXAMPLE 28

### 3-Amino-6-chloro-N-(6-phenylpyridin-2-yl)pyrazine-2-carboxamide

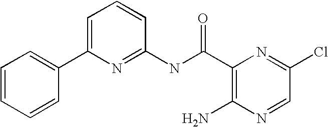

3-Amino-N-(6-bromopyridin-2-yl)-6-chloropyrazine-2-carboxamide was synthesized according to general procedure A using 6-bromopyridin-2-amine and 3-amino-6-chloropyrazine-2-carboxylic acid as starting materials.

3-Amino-6-chloro-N-(6-phenylpyridin-2-yl)pyrazine-2-carboxamide was synthesized according to general procedure C using 3-Amino-N-(6-bromopyridin-2-yl)-6-chloropyrazine-2-carboxamide and phenylboronic acid as starting materials. ¹H NMR (CDCl₃, 500 MHz) □ 10.09 (s, 1H), 8.44 (m, 2H), 8.05 (d, 1H), 7.81 (t, 1H), 7.55-7.42 (m, 5H). MS (ESI⁺) 326 (M⁺+1).

### EXAMPLE 29

### 3-Amino-6-chloro-N-(6-cyanopyridin-2-yl)pyrazine-2-carboxamide

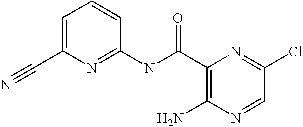

3-Amino-6-chloro-N-(6-cyanopyridin-2-yl)pyrazine-2-carboxamide was synthesized according to general procedure D using 3-Amino-N-(6-bromopyridin-2-yl)-6-chloropyrazine-2-carboxamide (see example 27) as starting material. ¹H NMR (CDCl₃, 500 MHz) □ 10.15 (s, 1H), 8.60 (d, 1H), 8.26 (s, 1H), 7.89 (t, 1H), 7.49 (d, 1H). MS (ESI⁺) 275 (M⁺+1).

### EXAMPLE 30

### 3-Amino-6-chloro-N-[6-(1H-imidazol-1-yl)pyridin-2-yl]pyrazine-2-carboxamide

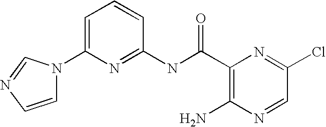

A mixture of 3-Amino-N-(6-bromopyridin-2-yl)-6-chloropyrazine-2-carboxamide (see example 27) (100 mg, 0.3 mmol), imidazole (41 mg, 0.6 mmol), copper iodide (6 mg, 0.032 mmol), 1,10-phenanthroline (10 mg, 0.06 mmol), and cesium carbonate (214 mg, 0.66 mmol) in MeOH (2 mL) was heated in a microwave at 180° C. for 10 min. The resulting mixture was cooled to room temperature, filtered through celite, and poured into a EtOAc/brine mixture. The two layers were separated and the aqueous was extracted with EtOAc (3×). The organics were combined, dried over sodium sulfate, filtered, and evaporated to dryness. The residue was purified by flash chromatography on silica gel eluting with a mixture of EtOAc/Hexane to yield 17 mg of 3-amino-6-chloro-N-[6-(1H-imidazol-1-yl)pyridin-2-yl]pyrazine-2-carboxamide. ¹H NMR (CDCl₃, 500 MHz) □ 10.03 (s, 1H), 8.40 (s, 1H), 8.30 (s, 1H), 8.28 (d, 1H), 7.92 (t, 1H), 7.71 (s, 1H), 7.24 (s, 1H), 7.17 (d, 1H). MS (ESI⁺) 316 (M⁺+1).

### EXAMPLE 31

### 3-Amino-N-2,4′-bipyridin-6-yl-6-chloropyrazine-2-carboxamide

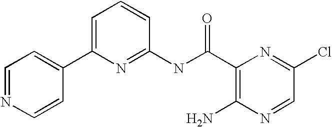

3-Amino-N-2,4′-bipyridin-6-yl-6-chloropyrazine-2-carboxamide was synthesized according to general procedure C using 3-Amino-N-(6-bromopyridin-2-yl)-6-chloropyrazine-2-carboxamide (see example 27) and pyridin-4-ylboronic acid as starting materials. ¹H NMR (CDCl₃, 500 MHz) □ 10.10 (s, 1H), 8.75 (d, 2H), 8.41 (d, 1H), 8.26 (s, 1H), 7.93 (d, 2H), 7.90 (t, 1H), 7.61 (d, 1H). MS (ESI⁺) 327 (M⁺+1).

### EXAMPLE 32

### 3-Amino-N-pyridin-2-ylpyrazine-2-carboxamide

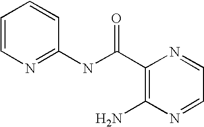

3-Amino-N-pyridin-2-ylpyrazine-2-carboxamide was synthesized according to general procedure A using pyridin-2-amine and 3-aminopyrazine-2-carboxylic acid as starting materials. ¹H NMR (D₄-MeOH, 500 MHz) □ 8.25-8.4 (m, 2H), 8.2 (s, 1H), 7.9 (s, 1H), 7.80-7.88 (m, 1H), 7.1-7.2 (m, 1H). MS (ESI⁺) 216 (M⁺+1).

### EXAMPLE 33

### 3-Amino-6-chloro-N-pyridin-2-ylpyrazine-2-carboxamide

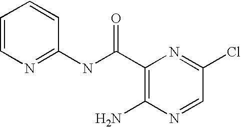

3-Amino-6-chloro-N-pyridin-2-ylpyrazine-2-carboxamide was synthesized according to general procedure A using pyridin-2-amine and 3-amino-6-chloropyrazine-2-carboxylic acid as starting materials. ¹H NMR (CDCl₃, 500 MHz) □ 10.1 (s, 1H), 8.5 (d, 1H), 8.4 (d, 1H), 8.3 (s, 1H), 7.8 (t, 1H), 7.15 (t, 1H). MS (ESI⁺) 250 (M⁺+1).

### EXAMPLE 34

### 3-Amino-6-chloro-5-methoxy-N-pyridin-2-ylpyrazine-2-carboxamide

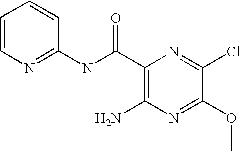

3-Amino-6-chloro-5-methoxy-N-pyridin-2-ylpyrazine-2-carboxamide was synthesized according to general procedure A using pyridin-2-amine and 3-amino-6-chloro-5-methoxypyrazine-2-carboxylic acid as starting materials. ¹H NMR (CDCl₃, 500 MHz) □ 9.9 (s, 1H), 8.4 (d, 1H), 8.3 (d, 1H), 7.8 (t, 1H), 7.10 (t, 1H). MS (ESI⁺) 279 (M⁺+1).

### EXAMPLE 35

### 6-Chloro-5-(dimethylamino)-N-pyridin-2-ylpyrazine-2-carboxamide

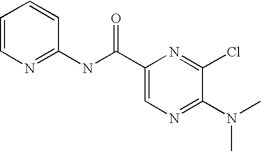

6-Chloro-5-(dimethylamino)-N-pyridin-2-ylpyrazine-2-carboxamide was synthesized according to general procedure A using pyridin-2-amine and 6-chloro-5-(dimethylamino)pyrazine-2-carboxylic acid as starting materials ¹H NMR (CDCl₃, 500 MHz) □ 9.8 (s, 1H), 8.9 (s, 1H), 8.3-8.45 (m, 2H), 7.8 (t, 1H), 7.10 (t, 1H). MS (ESI⁺) 275.9 (M⁺).

### EXAMPLE 36

### 3-Amino-6-chloro-5-(dimethylamino)-N-pyridin-2-ylpyrazine-2-carboxamide

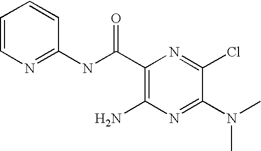

3-Amino-6-chloro-5-(dimethylamino)-N-pyridin-2-ylpyrazine-2-carboxamide was synthesized according to general procedure A using pyridin-2-amine and 3-amino-6-chloro-5-(dimethylamino)pyrazine-2-carboxylic acid as starting materials ¹H NMR (CDCl₃, 500 MHz) □ 9.7 (s, 1H), 8.3 (d, 1H), 8.27 (d, 1H), 7.7 (t, 1H), 7.05 (t, 1H), 4.2-4.4 (m, 1H), 1.25 (d, 6H).

### EXAMPLE 37

### 3-Amino-6-methyl-N-pyridin-2-ylpyrazine-2-carboxamide

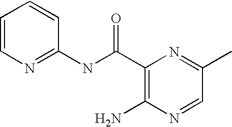

3-Amino-6-methyl-N-pyridin-2-ylpyrazine-2-carboxamide was synthesized according to general procedure A using pyridin-2-amine and 3-amino-6-methylpyrazine-2-carboxylic acid as starting materials. ¹H NMR (CDCl₃, 500 MHz) □ 10.35 (s, 1H), 8.4 (d, 1H), 8.25 (d, 1H), 8.1 (s, 1H), 7.8 (t, 1H), 7.1 (t, 1H), 2.45 (s, 3H).

### EXAMPLE 38

### 3-Amino-N-pyridin-2-yl-5-(trifluoromethyl)pyrazine-2-carboxamide

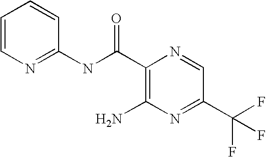

3-Amino-N-pyridin-2-yl-5-(trifluoromethyl)pyrazine-2-carboxamide was synthesized according to general procedure A using pyridin-2-amine and 3-amino-5-trifluoromethylpyrazine-2-carboxylic acid as starting materials. ¹H NMR (CDCl₃, 500 MHz) □ 10.3 (s, 1H), 8.4 (d, 1H), 8.3 (d, 1H), 8.2 (s, 1H), 7.8 (t, 1H), 7.1 (t, 1H). MS (ESI⁺) 284.0 (M⁺1).

### EXAMPLE 39

### 3-Amino-N-pyridin-2-yl-6-(trifluoromethyl)pyrazine-2-carboxamide

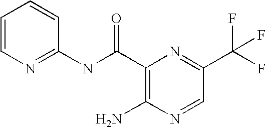

3-Amino-N-pyridin-2-yl-6-(trifluoromethyl)pyrazine-2-carboxamide was synthesized according to general procedure A using pyridin-2-amine and 3-amino-6-trifluoromethylpyrazine-2-carboxylic acid as starting materials. MS (ESI⁺) 306.0 (M⁺23).

### EXAMPLE 40

### 3-Amino-N-pyridin-2-yl-5,6,7,8-tetrahydroquinoxaline-2-carboxamide

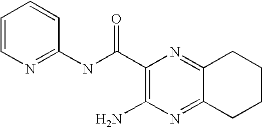

3-Amino-N-pyridin-2-yl-5,6,7,8-tetrahydroquinoxaline-2-carboxamide was synthesized according to general procedure A using pyridin-2-amine and 3-amino-5,6,7,8-tetrahydroquinoxaline-2-carboxylic acid as starting materials. ¹H NMR (CDCl₃, 500 MHz) □10.35 (s, 1H), 8.3-8.4 (m, 2H), 7.8 (t, 1H), 7.1 (t, 1H), 2.7-2.8 (m, 4H), 1.9-2.0 (m, 4H). MS (ESI⁺) 270.0 (M⁺1).

### EXAMPLE 41

### 3-Amino-5-chloro-6-phenyl-N-pyridin-2-ylpyrazine-2-carboxamide

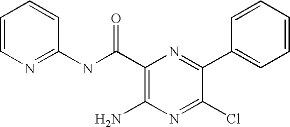

3-Amino-5-chloro-6-phenyl-N-pyridin-2-ylpyrazine-2-carboxamide was synthesized according to general procedure A using pyridin-2-amine and 3-amino-5-chloro-6-phenylpyrazine-2-carboxylic acid as starting materials. MS (ESI⁺) 326.0 (M⁺1).

### EXAMPLE 42

### 3-Amino-N-pyridin-2-ylquinoxaline-2-carboxamide

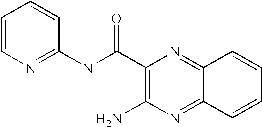

3-Amino-N-pyridin-2-ylquinoxaline-2-carboxamide was synthesized according to general procedure A using pyridin-2-amine and 3-aminoquinoxaline-2-carboxylic acid as starting materials. MS (ESI⁺) 266.0 (M⁺1).

### EXAMPLE 43

### 3-Amino-6-chloro-5-(ethylamino)-N-pyridin-2-ylpyrazine-2-carboxamide

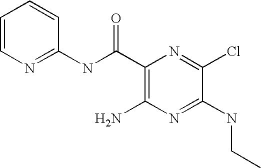

3-Amino-6-chloro-5-(ethylamino)-N-pyridin-2-ylpyrazine-2-carboxamide was synthesized according to general procedure A using pyridin-2-amine and 3-amino-6-chloro-5-(ethylamino)pyrazine-2-carboxylic acid as starting materials. ¹H NMR (CDCl₃, 500 MHz) □ 9.7 (s, 1H), 8.35-8.2 (m, 2H), 7.7 (t, 1H), 7.0 (t, 1H), 5.4 (s, 1H), 3.5 (q, 2H), 1.3 (t, 3H). MS (ESI⁺) 293.0 (M⁺).

### EXAMPLE 44

### 3-Amino-6-ethyl-N-pyridin-2-ylpyrazine-2-carboxamide

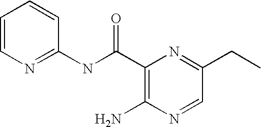

3-Amino-6-ethyl-N-pyridin-2-ylpyrazine-2-carboxamide was synthesized according to general procedure A using pyridin-2-amine and 3-amino-6-ethylpyrazine-2-carboxylic acid as starting materials. MS (ESI⁺) 244.0 (M⁺1).

### EXAMPLE 45

### 6-chloro-N-pyridin-2-ylpyrazine-2-carboxamide

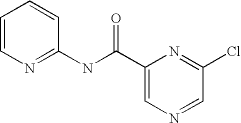

6-Chloro-N-pyridin-2-ylpyrazine-2-carboxamide was synthesized according to general procedure A using pyridin-2-amine and 6-chloropyrazine-2-carboxylic acid as starting materials. ¹H NMR (CDCl₃, 500 MHz) □ 10.0 (s, 1H), 9.4 (s, 1H), 8.8 (s, 1H), 8.4 (d, 2H), 7.8 (t, 2H), 7.1 (t, 1H). MS (ESI⁺) 235.4 (M⁺1).

### EXAMPLE 46

### 3-Amino-5-(isopropylamino)-N-pyridin-2-ylpyrazine-2-carboxamide

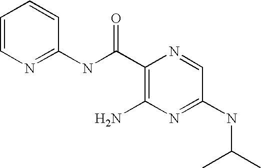

3-Amino-5-(isopropylamino)-N-pyridin-2-ylpyrazine-2-carboxamide was synthesized according to general procedure A using pyridin-2-amine and 3-amino-5-(isopropylamino)pyrazine-2-carboxylic acid as starting materials. ¹H NMR (d₆-DMSO, 500 MHz) □ 9.8 (s, 1H), 8.3 (d, 1H), 8.2 (d, 1H), 7.8 (t, 1H), 7.7 (br s, 1H), 7.0 (t, 1H), 4.0-4.2 (m, 1H), 1.1 (d, 6H). MS (ESI⁺) 273.5 (M⁺).

### EXAMPLE 47

### 3-Amino-6-butyl-N-pyridin-2-ylpyrazine-2-carboxamide

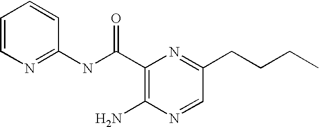

3-Amino-6-butyl-N-pyridin-2-ylpyrazine-2-carboxamide was synthesized according to general procedure A using pyridin-2-amine and 3-amino-6-n-butylpyrazine-2-carboxylic acid as starting materials. MS (ESI⁺) 272.6 (M⁺1).

### EXAMPLE 48

### 3-Amino-5,6-dimethyl-N-pyridin-2-ylpyrazine-2-carboxamide

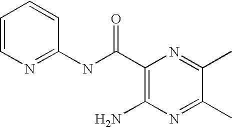

3-Amino-5,6-dimethyl-N-pyridin-2-ylpyrazine-2-carboxamide was synthesized according to general procedure A using pyridin-2-amine and 3-amino-5,6-dimethylpyrazine-2-carboxylic acid as starting materials. ¹H NMR (CDCl₃, 500 MHz) □ 10.3 (s, 1H), 8.3-8.4 (m, 2H), 7.7 (t, 1H), 7.0 (t, 1H), 2.4 (s, 6H).

### EXAMPLE 49

### N-Pyridin-2-ylquinoxaline-2-carboxamide

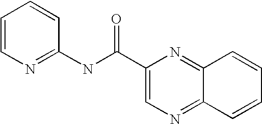

N-Pyridin-2-ylquinoxaline-2-carboxamide was synthesized according to general procedure A using pyridin-2-amine and quinoxaline-2-carboxylic acid as starting materials. MS (ESI⁺) 251.6 (M⁺1).

### EXAMPLE 50

### 3-Amino-6-methyl-N-(6-methylpyridin-2-yl)pyrazine-2-carboxamide

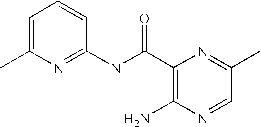

3-Amino-6-methyl-N-(6-methylpyridin-2-yl)pyrazine-2-carboxamide was synthesized according to general procedure A using pyridin-2-amine and 3-amino-6-methylpyrazine-2-carboxylic acid as starting materials. MS (ESI⁺) 44.1 (M⁺1).

### EXAMPLE 51

### 3-Amino-5,6-dimethyl-N-(6-methylpyridin-2-yl)pyrazine-2-carboxamide

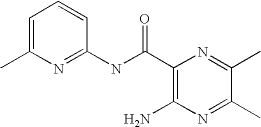

3-Amino-5,6-dimethyl-N-(6-methylpyridin-2-yl)pyrazine-2-carboxamide was synthesized according to general procedure A using pyridin-2-amine and 3-amino-5,6-dimethylpyrazine-2-carboxylic acid as starting materials. MS (ESI⁺) 258.2 (M⁺1).

### EXAMPLE 52

### 3-Amino-6-chloro-N-{6-[(trimethylsilyl)ethynyl]pyridin-2-yl}pyrazine-2-carboxamide

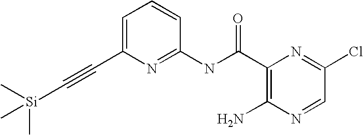

To a solution of 6-bromopyridin-2-amine (4 g, 23 mmol), trimethylsilyl acetylene (4.5 mL, 32 mmol), and CuI (88 mg, 0.46 mmol) in Et₂NH (75 mL) was added PdCl₂(Ph₃P)₂ (650 mg, 0.92 mmol). The reaction was stirred at rt for 18 h and then concentrated in vacuo. The crude residue was purified by silica gel chromatography (EtOAc/hexanes) to give 6-[(trimethylsilyl)-ethynyl]pyridin-2-amine as an oil.

3-amino-6-chloro-N-{6-[(trimethylsilyl)ethynyl]pyridin-2-yl}pyrazine-2-carboxamide was synthesized according to general procedure A using 6-[(trimethylsilyl)ethynyl]pyridin-2-amine and 3-amino-6-chloropyrazine-2-carboxylic acid as starting materials. ¹H NMR (CDCl₃, 500 MHz) □ 10.1 (s, 1H), 8.3 (d, 1H), 8.2 (s, 1H), 7.7 (t, 1H), 7.3 (t, 1H), 0.3 (s, 9H). MS (ESI⁺) 346.1 (M⁺).

### EXAMPLE 53

### 6-Chloro-N-{6-[(trimethylsilyl)ethynyl]pyridin-2-yl}pyridine-2-carboxamide

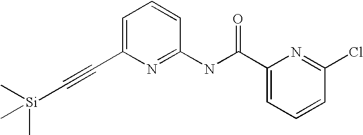

6-Chloro-N-{6-[(trimethylsilyl)ethynyl]pyridin-2-yl}pyridine-2-carboxamide was synthesized according to general procedure A using 6-[(trimethylsilyl)ethynyl]pyridin-2-amine and 6-chloropyradin-2-carboxylic acid as starting materials. ¹H NMR (CDCl₃, 500 MHz) □ 10.7 (s, 1H), 8.5 (d, 1H), 8.1 (s, 1H), 7.8 (t, 1H), 7.7 (t, 1H), 7.4 (d, 1H), 7.28 (d, 1H), 2.6 (s, 3H), 0.4 (s, 9H). MS (ESI⁺) 310.3 (M⁺1).

### EXAMPLE 54

### 3-Amino-6-chloro-N-(6-ethynylpyridin-2-yl)pyrazine-2-carboxamide

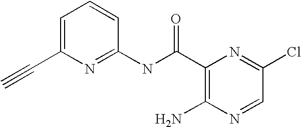

To a solution of 3-amino-6-chloro-N-{6-[(trimethylsilyl)ethynyl]pyridin-2-yl}pyrazine-2-carboxamide (0.57 g, 1.84 mmol) in THF (10 mL) at 0 C was added TBAF (2.8 mL, 2.8 mmol). When starting material was consumed as judged by TLC, the reaction was concentrated in vacuo and purified by silica gel chromatography (EtOAc/hexanes) to give 3-amino-6-chloro-N-(6-ethynylpyridin-2-yl)pyrazine-2-carboxamide as a pale yellow solid. MS (ESI⁺) 273.9 (M⁺).

### EXAMPLE 55

### N-(6-ethynylpyridin-2-yl)-6-methylpyridine-2-carboxamide

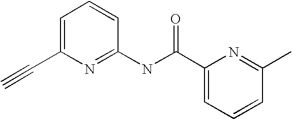

N-(6-ethynylpyridin-2-yl)-6-methylpyridine-2-carboxamide was prepared following the same general procedure as described in Example 54. ¹H NMR (CDCl₃, 500 MHz) □ 10.6 (s, 1H), 8.5 (d, 1H), 8.1 (d, 1H), 7.75-7.9 (m, 2H), 7.4 (d, 1H), 7.28 (d, 1H), 3.2 (s, 1H), 2.6 (s, 3H). MS (ESI⁺) 238.1 (M⁺).

### EXAMPLE 56

### N-(6-ethylpyridin-2-yl)-6-methylpyridine-2-carboxamide

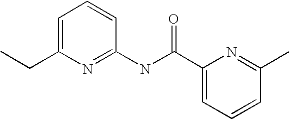

A mixture of N-(6-ethynylpyridin-2-yl)-6-methylpyridine-2-carboxamide (100 mg) and 10% Pd/C and EtOAc was stirred under a balloon of H2 for 16 h. The catalyst was removed by filtration through silica gel and concentrated to give N-(6-ethylpyridin-2-yl)-6-methylpyridine-2-carboxamide as a colorless solid. MS (ESI⁺) 242.2 (M⁺1).

### EXAMPLE 57

### N-(6-bromopyridin-2-yl)-6-methylpyridine-2-carboxamide

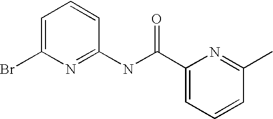

N-(6-bromopyridin-2-yl)-6-methylpyridine-2-carboxamide was synthesized according to general procedure A using 6-bromopyridin-2-amine and 6-methylpyridine-2-carboxylic acid as starting materials. ¹H NMR (CDCl₃, 500 MHz) □ 10.6 (s, 1H), 8.6 (d, 1H), 8.1 (d, 1H), 7.8 (d, 1H), 7.6 (d, 1H), 7.4 (d, 1H), 7.26 (d, 1H), 2.6 (s, 3H). MS (ESI⁺) 292.2 (M⁺).

### EXAMPLE 58

### 6-Methyl-N-(6-phenylpyridin-2-yl)pyridine-2-carboxamide

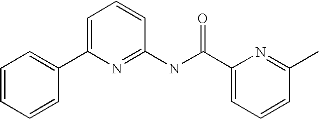

6-Methyl-N-(6-phenylpyridin-2-yl)pyridine-2-carboxamide was synthesized according to general procedure C using N-(6-bromopyridin-2-yl)-6-methylpyridine-2-carboxamide and phenylboronic acid as starting materials. MS (ESI⁺) 290.5 (M⁺1).

### EXAMPLE 59

### N-2,3′-bipyridin-6-yl-6-methylpyridine-2-carboxamide

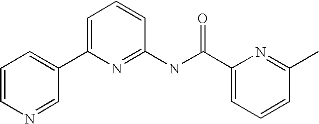

N-2,3′-bipyridin-6-yl-6-methylpyridine-2-carboxamide was synthesized according to general procedure C using N-(6-bromopyridin-2-yl)-6-methylpyridine-2-carboxamide and 3-pyridylboronic acid as starting materials. MS (ESI⁺) 291.5 (M⁺1).

### EXAMPLE 60

### N-(6-cyanopyridin-2-yl)-6-methylpyridine-2-carboxamide

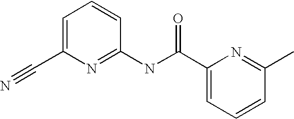

N-(6-cyanopyridin-2-yl)-6-methylpyridine-2-carboxamide was synthesized according to general procedure D using N-(6-bromopyridin-2-yl)-6-methylpyridine-2-carboxamide and NaCN as starting materials. MS (ESI⁺) 239.3 (M⁺1).

### EXAMPLE 61

### N-[6-(1H-imidazol-1-yl)pyridin-2-yl]-6-methylpyridine-2-carboxamide

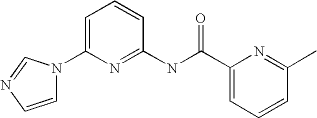

N-[6-(1H-imidazol-1-yl)pyridin-2-yl]-6-methylpyridine-2-carboxamide was synthesized as described in example 30 using N-(6-bromopyridin-2-yl)-6-methylpyridine-2-carboxamide and imidazole as starting materials. ¹H NMR (CDCl₃, 500 MHz) □ 10.5 (s, 1H), 8.4 (d, 2H), 8.1 (d, 1H), 7.9 (d, 1H), 7.8 (d, 1H), 7.7 (s, 1H), 7.4 (d, 1H), 7.2 (s, 1H), 7.1 (d, 1H), 2.7 (s, 3H). MS (ESI⁺) 280.5 (M⁺1).

### EXAMPLE 62

### 3-Amino-6-cyano-N-pyridin-2-ylpyrazine-2-carboxamide

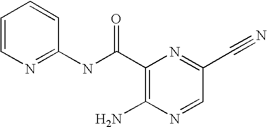

3-Amino-6-cyano-N-pyridin-2-ylpyrazine-2-carboxamide was synthesized according to general procedure A using pyridin-2-amine and 3-amino-6-cyanopyrazine-2-carboxylic acid as starting materials. ¹H NMR (CDCl₃, 500 MHz) □ 10.1 (s, 1H), 8.8 (br s, 1H), 8.5 (s, 1H), 8.4 (s, 1H), 8.3 (d, 1H), 7.8 (t, 1H), 7.1 (t, 1H), 5.9 (br s, 1H). MS (ESI⁺) 241.2 (M⁺1).

### EXAMPLE 63

### 3-Amino-6-chloro-N-(6-methylpyridin-2-yl)pyrazine-2-carboxamide

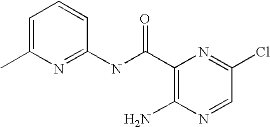

3-Amino-6-chloro-N-(6-methylpyridin-2-yl)pyrazine-2-carboxamide was synthesized according to general procedure A using 6-methylpyridin-2-amine and 3-amino-6-chloropyrazine-2-carboxylic acid as starting materials. MS (ESI⁺) 264.4 (M⁺).

### EXAMPLE 64

### 3-Hydroxy-6-methyl-N-(6-methylpyridin-2-yl)pyridine-2-carboxamide

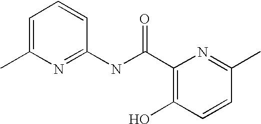

3-Hydroxy-6-methyl-N-(6-methylpyridin-2-yl)pyridine-2-carboxamide was synthesized according to general procedure A using 6-methylpyridin-2-amine and 3-hydroxy-6-methylpyridine-2-carboxylic acid as starting materials. MS (ESI⁺) 244.3 (M⁺1).

### EXAMPLE 65

### Allyl(6-methyl-2-{[(6-methylpyridin-2-yl)amino]carbonyl}pyridin-3-yl)carbamate

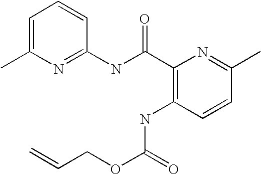

Allyl(6-methyl-2-{[(6-methylpyridin-2-yl)amino]carbonyl}pyridin-3-yl)carbamate was synthesized according to general procedure A using 6-methylpyridin-2-amine and 3-{[(allyloxy)carbonyl]amino}-6-methylpyridine-2-carboxylic acid as starting materials. ¹H NMR (CDCl₃, 500 MHz) □ 11.3 (s, 1H), 10.7 (s, 1H), 8.8 (d, 1H), 8.2 (d, 1H), 7.7 (t, 1H), 7.4 (d, 1H), 6.9 (d, 1H), 5.9-6.0 (m, 1H), 5.4 (d, 1H), 5.3 (d, 1H), 4.7 (d, 2H), 2.6 (s, 3H), 2.55 (s, 3H). MS (ESI⁺) 327.6 (M⁺1).

### EXAMPLE 66

### 3-Amino-6-methyl-N-(6-methylpyridin-2-yl)pyridine-2-carboxamide

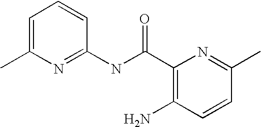

To a solution of allyl(6-methyl-2-{[(6-methylpyridin-2-yl)amino]carbonyl}pyridin-3-yl)carbamate (370 mg, 1.1 mmol) and PhSiH₃ (245 mg, 2.3 mmol) in THF (5 mL) was added Pd(Ph₃P)₄ (130 mg, 0.11 mmol). When starting material was consumed as judged by TLC analysis, the reaction was quenched with H₂O (0.1 mL) and concentrated in vacuo to give 3-amino-6-methyl-N-(6-methylpyridin-2-yl)pyridine-2-carboxamide as an off-white solid. MS (ESI⁺) 243.5 (M⁺1).

### EXAMPLE 67

### 4,6-Dichloro-N-(6-methylpyridin-2-yl)pyridine-2-carboxamide

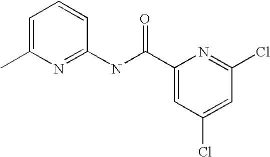

4,6-Dichloro-N-(6-methylpyridin-2-yl)pyridine-2-carboxamide was synthesized according to general procedure A using 6-methylpyridin-2-amine and 4,6-dichloropyridine-2-carboxylic acid as starting materials. MS (ESI⁺) 283.2 (M⁺1).

### EXAMPLE 68

### 6-Methyl-N-(6-methylpyridin-2-yl)-3-(pyridin-3-ylamino)pyridine-2-carboxamide

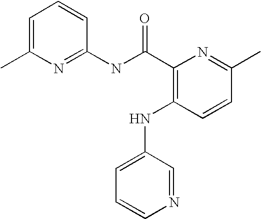

A Mixture of 3-bromopyridine (112 mg, 0.71 mmol), 3-amino-6-methyl-N-(6-methylpyridin-2-yl)pyridine-2-carboxamide (115 mg, 0.48 mmol), Pd₂(dba)₃ (44 mg, 0.04 mmol), BINAP (59 mg, 0.10 mmol), and sodium tert-butoxide (91 mg, 0.95 mmol) in toluene (3 mL) was placed in a sealed tube and heated in a microwave (Personal Chemistry, Model: Smith Creator) for 15 min at 140° C. The reaction mixture was filtered through a celite pad, rinsed with EtOAc and concentrated in vacuo. The residue was purified by flash chromatography on silica gel (ethyl acetate/hexanes) to afford 6-methyl-N-(6-methylpyridin-2-yl)-3-(pyridin-3-ylamino)pyridine-2-carboxamide as a pale yellow solid. ¹H NMR (CDCl₃, 500 MHz) □ 10.8 (s, 1H), 10.1 (s, 1H), 8.8 (s, 1H), 8.4 (s, 1H), 8.2 (d, 1H), 7.7 (t, 1H), 7.51 (d, 2H), 7.27-735 (m, 1H), 7.2 (d, 1H), 6.9 (d, 1H), 2.6 (s, 3H), 2.58 (s, 3H). MS (ESI⁺) 320.2 (M⁺1).

Other variations or modifications, which will be obvious to those skilled in the art, are within the scope and teachings of this invention. This invention is not to be limited except as set forth in the following claims.

What is claimed is:

1. A compound represented by Formula (I): 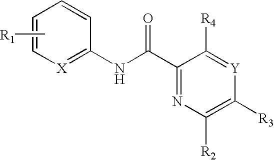
or a pharmaceutically acceptable salt thereof wherein:
X is —N—, or —C—
Y is —N—, —C—, or C-halogen.
R₁ is selected from:
1) hydrogen,
2) C₁₋₁₀alkyl,
3) C₂₋₁₀alkenyl,
4) C₂₋₁₀alkynyl
5) C₃₋₁₀cycloalkyl,
6) heterocyclyl,
7) aryl,
8) heteroaryl,
9) —NR^{d}R^{e},
10) —CO₂R^{d},
11) —OR^{d},
12) —CN, and
13) halogen,
where alkyl, alkenyl, alkynyl, cycloalkyl and heterocyclyl are optionally substituted with 1, 2, 3 or 4 substituents selected from R^{a}, and where aryl and heteroaryl are optionally substituted with 1, 2, 3, 4 or 5 substituents independently selected from R^{b};
R₂ is selected from:
1) hydrogen,
2) C₁₋₁₀alkyl,
3) C₂₋₁₀alkenyl,
7) C₂₋₁₀alkynyl,
8) C₃₋₁₀cycloalkyl,
9) heterocyclyl,
7) aryl,
8) —CN,
9) halogen,
10) —OR^{d}, and
11) heteroaryl,
where alkyl, alkenyl and alkynyl, cycloalkyl and heterocyclyl, aryl, and heteroaryl are optionally substituted with 1, 2, 3, 4 or 5 five substituents independently selected from R^{b};
R₃ is selected from:
1) aryl,
2) NR^{d}R^{e},
3) halogen,
4) C₁₋₁₀alkyl,
5) —OR^{d},
6) hydrogen, and
7) —SR^{d},
where alkyl are optionally substituted with 1, 2, 3, 4 or 5 substituents selected from R^{a};
R² and R³ may be joined together with the atoms to which they are attached to form a saturated or unsaturated ring of 4, 5, 6 or 7 members containing 0, 1 or 2 heteroatoms independently selected from oxygen, sulfur and nitrogen;
R₄ is selected from:
1) aryl,
2) heteroaryl,
3) —NR^{d}R^{e},
4) halogen,
5) —OR^{d},
6) hydrogen, and
7) SR^{d};
where aryl and heteroaryl are optionally substituted with 1, 2, 3, 4 or 5 substituents independently selected from R^{b};
R^{a} is selected from:
1) hydrogen,
2) —OR^{d},
3) —NO₂,
4) halogen,
5) —S(O)ₘR^{d},
6) —SR^{d},
7) —S(O)ₘNR^{d}R^{e},
8) —NR^{d}R^{e},
9) —C(O)R^{d},
10) —CO₂R^{d},
11) —OC(O)R^{d},
12) —CN,
13) —SiR^{c}R^{d}R^{e},
14) —C(O)NR^{d}R^{e},
15) —NR^{d}C(O)R^{e},
16) —OC(O)NR^{d}R^{e},
17) —NR^{d}C(O)OR^{e},
18) —NR^{d}C(O)NR^{d}R^{e},
19) —CR^{d}(N—OR^{e}),
20) CF₃, and
21) —OCF₃;
R^{b} is selected from:
1) R^{a},
2) C₁₋₁₀alkyl,
3) C₂₋₁₀ alkenyl,
4) C₂₋₁₀ alkynyl,
5) C₃₋₁₀cycloalkyl,
6) heterocyclyl,
7) aryl, and
8) heteroaryl,
where alkyl, alkenyl, alkynyl, cycloalkyl, heterocyclyl, aryl, heteroaryl are optionally substituted with 1, 2, 3, 4 or 5 substituents independently selected from R^{c};
R^{c} is selected from:
1) halogen,
2) amino,
3) carboxy,
4) cyano,
5) C₁₋₄alkyl,
6) C₁₋₄alkoxy,
7) aryl,
58) arylC₁₋₄alkyl,
9) heteroaryl,
10) hydroxy,
11) CF₃, and
12) aryloxy;
R^{d} and R^{e} are independently selected from R^{a}, C₁₋₁₀alkyl,
C₂₋₁₀alkenyl, C₂₋₁₀alkynyl and Cy, where alkyl, alkenyl, alkynyl and Cy are optionally substituted with 1, 2, 3, 4 or 5 substituents independently selected from R^{c};
or R^{d} and R^{e} together with the atoms to which they are attached form a saturated or unsaturated ring of 4, 5, 6 or 7 members containing 0, 1 or 2 heteroatoms independently selected from oxygen, sulfur and nitrogen;
Cy is independently selected from cycloalkyl, heterocyclyl, aryl, or heteroaryl; and
m is 1 or 2.

2. A compound according to claim 1 wherein:
R₁ is selected from:
1) hydrogen,
2) C₁₋₆alkyl,
3) C₂₋₆alkenyl,
4) C₂₋₆alkylyl,
5) C₃₋₆cycloalkyl,
6) heterocyclyl,
7) aryl,
8) heteroaryl,
9) —NR^{d}R^{e},
10) —OR^{d},
11) —CO₂R^{d},
10) —CN,
12) halogen;
where alkyl, alkenyl, alkylyl, cycloalkyl and heterocyclyl are optionally substituted with one to four substituents selected from R^{a}, and where aryl and heteroaryl are optionally substituted with 1, 2 or 3 substituents independently selected from R^{b};
R₂ is selected from:
1) hydrogen,
2) C₁₋₆alkyl,
3) C₂₋₆alkenyl,
4) C₃₋₆cycloalkyl,
5) aryl,
6) heteroaryl,
7) —CN,
8) —OR^{d}, and
9) halogen,
where alkyl, alkenyl, cycloalkyl, aryl and heteroaryl are optionally substituted with 1, 2 or 3 substituents independently selected from R^{b};
R₃ is selected from:
1) hydrogen,
2) C₁₋₆alkyl,
3) aryl,
4) —NR^{d}R^{e},
5) —OR^{d},
6) —SR^{d},
7) halogen;
wherein alkyl is optionally substituted with 1, 2 or 3 substituents independently selected from R^{a};
R² and R³ may be joined so that together with the atoms to which R² and R³ are attached there is formed a cyclohexyl or phenyl ring;
R⁴ is selected from:
1) hydrogen,
2) aryl,
3) heteroaryl,
4) —NHR^{d},
5) —OR^{d},
6) —SR^{d},
7) halogen;
where aryl and heteroaryl are optionally substituted with 1, 2 or 3 substituents independently selected from R^{b};
R^{a} is selected from:
1) hydrogen,
2) —OR^{d},
3) halogen,
4) —NR^{d}R^{e},
5) —CN,
6) CO₂R^{d},
7) CF₃
R^{b} is selected from:
1) R^{a},
2) C₁₋₃ alkyl
where alkyl are optionally substituted with 1, 2 or 3 substituents independently selected from R^{c};
R^{c} is selected from:
1) hydrogen,
2) carboxy
3) C₁₋₃alkyl,
R^{d} and R^{e} are independently selected from R^{a}, C₁₋₄alkyl, cycloalkyl, aryl, or heteroaryl, where alkyl, cycloalkyl, aryl, or heteroaryl are optionally substituted with 1, 2 or 3 substituents independently selected from R^{c},
or R^{d} and R^{e} together with the atoms to which they are attached form a saturated or unsaturated ring of 4, 5, 6 or 7 members containing 0, 1 or 2 heteroatoms independently selected from oxygen, sulfur and nitrogen.

3. A compound according to claim 2 wherein:
R^{a} is selected from:
1) hydrogen,
2) —CN,
3) halogen;
R^{b} is selected from R^{a}.

4. A compound according to claim 3 wherein:
R₁ is selected from:
10) hydrogen,
11) methyl, ethyl
12) —C(O)—O—CH₃,
13) pyridinyl,
14) —CN,
15) imidazolyl,
16) chloro, bromo,
17) —CH—CH, and
18) hydroxyl,
wherein alkyl and heterocyclyl are optionally substituted with 1 or 2 substituents selected from R^{a}, and where heteroaryl are optionally substituted with 1 or 2 substituents independently selected from R^{b}.

5. A compound according to claim 3 wherein:
R₂ is selected from:
9) hydrogen,
10) Phenyl, optionally mono or di-substituted with a substituent selected from halo, —CH₃ and cyano,
11) CH₃, ethyl, butyl,
12) Bromo, chloro,
13) —CN,
14) —OCH₃,
15) pyridinyl, thienyl, and
16) —CF₃,
where alkyl, alkenyl, cycloalkyl, aryl and heteroaryl are optionally substituted with 1, 2 or 3 substituents independently selected from R^{b}.

6. A compound according to claim 3 wherein:
R₃ is selected from:
1) hydrogen,
2) —N(CH₃)CH₃,
3) CH₃,
4) piperidinyl,
5) —S—CH₃,
6) —NCH₂CH₃,
7) —OCH₃,
8) —N—CH₂-furanyl,
9) —N—CH(CH₃)₂,
10) CF₃,
11) phenyl,
12) chloro, and
13) —NH₂,
wherein alkyl is optionally substituted with 1, 2 or 3 substituents independently selected from R^{a}.

7. A compound according to claim 3 wherein:
R₂ and R₃ together with the atoms to which they are attached form a ring selected from cyclohexyl and phenyl.

8. A compound according to claim 3 wherein:
R₄ is selected from:
1) hydrogen,
2) —NH₂,
3) hydroxyl,
4) —N-pyridyl,
5) —S—CH₃,
6) —N(CH₃)₂,
7) —N—C(O)—O—CH₂C═CH₂.
where aryl and heteroaryl are optionally substituted with 1, 2 or 3 substituents independently selected from R^{b}.

9. A compound according to claim 3 of Formula (Ia): 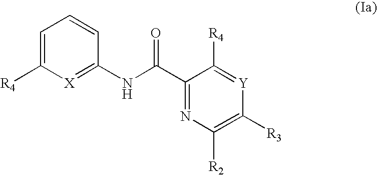
wherein
R₁ is selected from:
1) hydrogen,
2) methyl, ethyl
3) —C(O)—O—CH₃,
4) pyridinyl,
5) —CN,
6) imidazolyl,
7) chloro, bromo,
8) —CH≡CH—Si(CH₃)₃,
9) —CH≡CH, and
10) hydroxyl;
R₂ is selected from:
1) hydrogen,
2) Phenyl, optionally mono or di-substituted with a substituent selected from halo, —CH₃ and cyano,
3) CH₃, ethyl, butyl,
4) Bromo, chloro,
5) —CN,
6) —OCH₃,
7) pyridinyl, thienyl, and
8) —CF₃;
R₃ is selected from:
1) hydrogen,
2) —N(CH₃)CH₃,
3) CH₃,
4) piperidinyl,
5) —S—CH₃,
6) —NCH₂CH₃,
7) —OCH₃,
8) —N—CH₂-furanyl,
9) —N—CH(CH₃)₂,
10) CF₃,
11) phenyl,
12) chloro, and
13) —NH₂;
R₂ and R₃ together with the atoms to which they are attached form a ring selected from cyclohexyl and phenyl; and
R₄ is selected from:
1) hydrogen,
2) —NH₂,
3) hydroxyl,
4) —N-pyridyl,
5) —S—CH₃,
6) —N(CH₃)₂,
7) —N—C(O)—O—CH₂C═CH₂.

10. A compound according to claim 9 wherein
R₃ is hydrogen or methyl.

11. A compound according to claim 9 wherein
R₄ is hydroxyl, —NH₂ or —NH-aryl.

12. A compound according to claim 9 wherein
R₂ is halo or methyl.

13. A compound according to claim 9 wherein
R₁ is hydrogen or methyl.

14. A compound according to claim 9 wherein
R₁ is hydrogen or methyl;
R₂ is halo or methyl;
R₃ is hydrogen or methyl; and
R₄ is hydroxyl, —NH₂ or —NH-aryl.

15. A compound selected from: 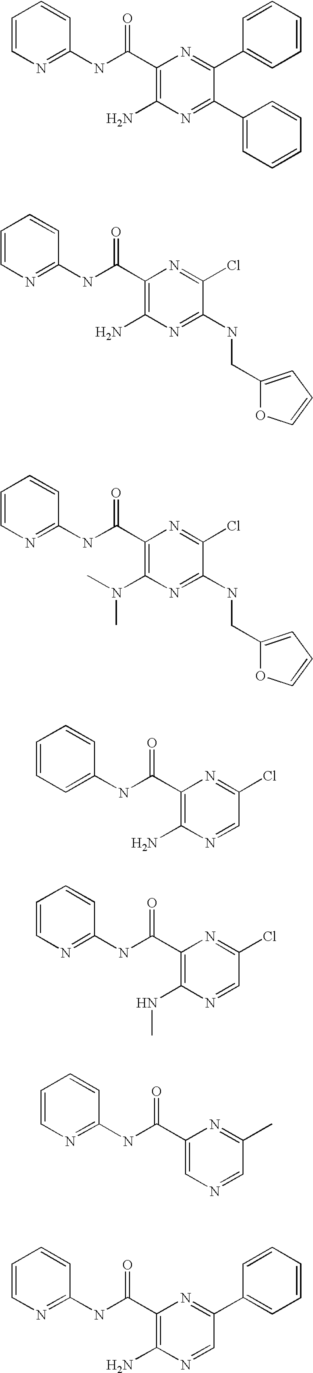 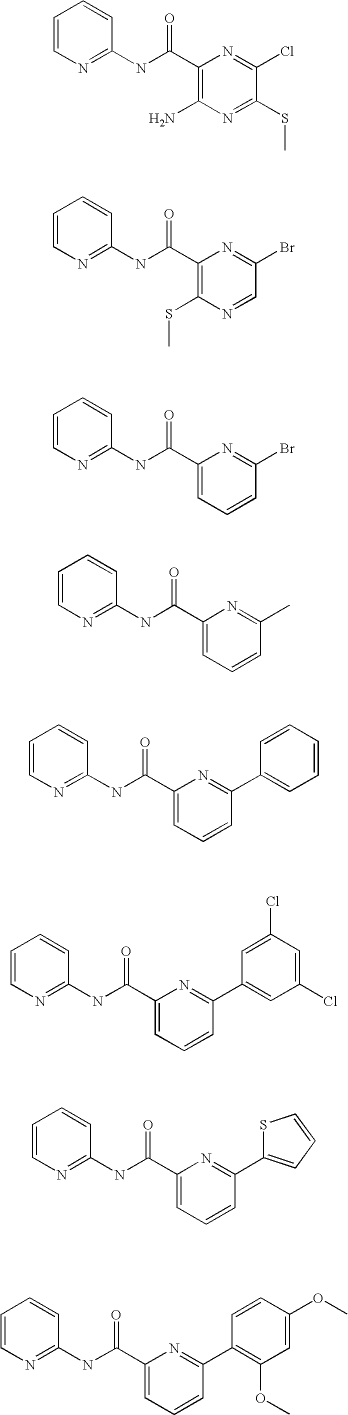 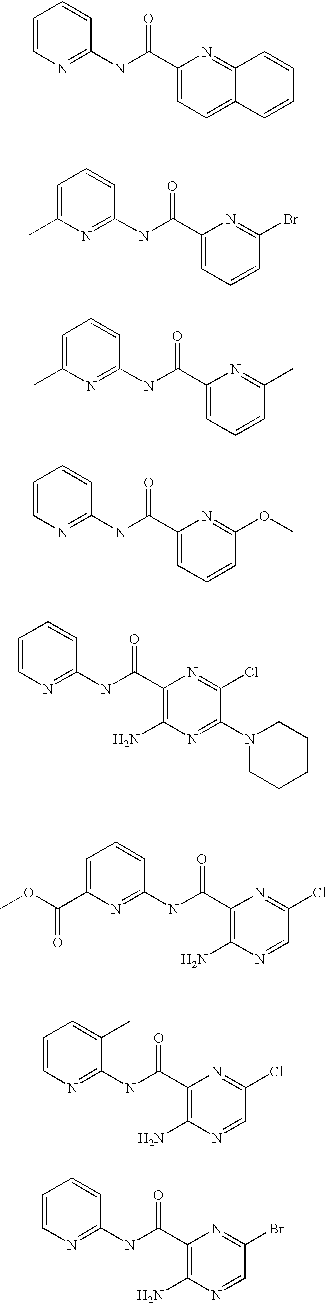 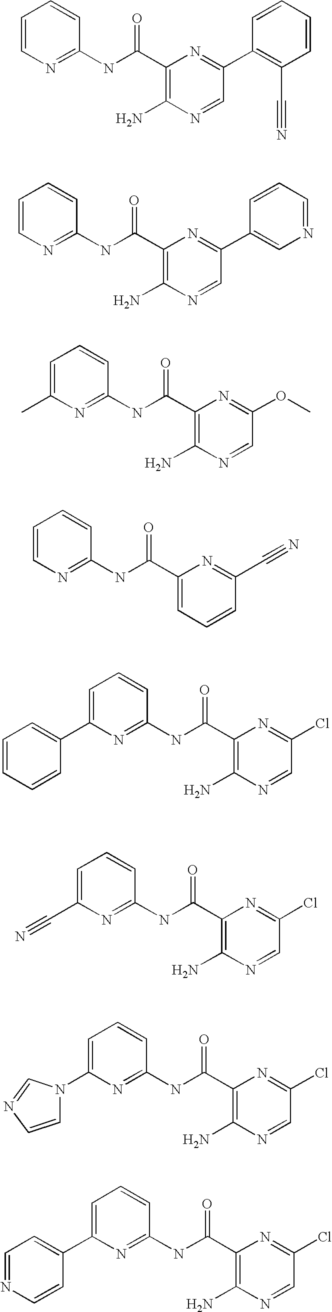 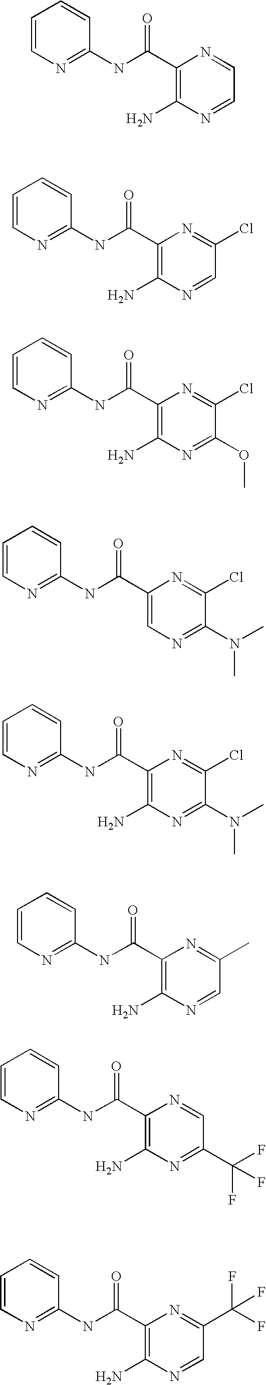 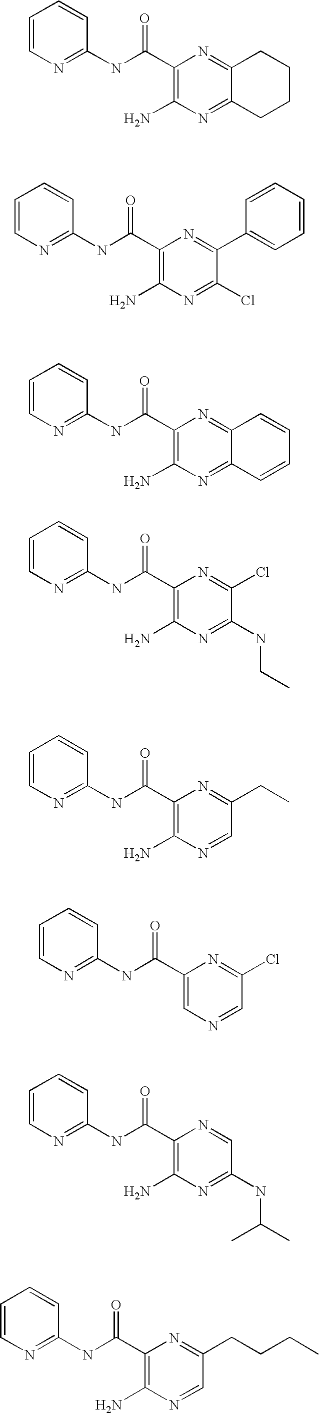 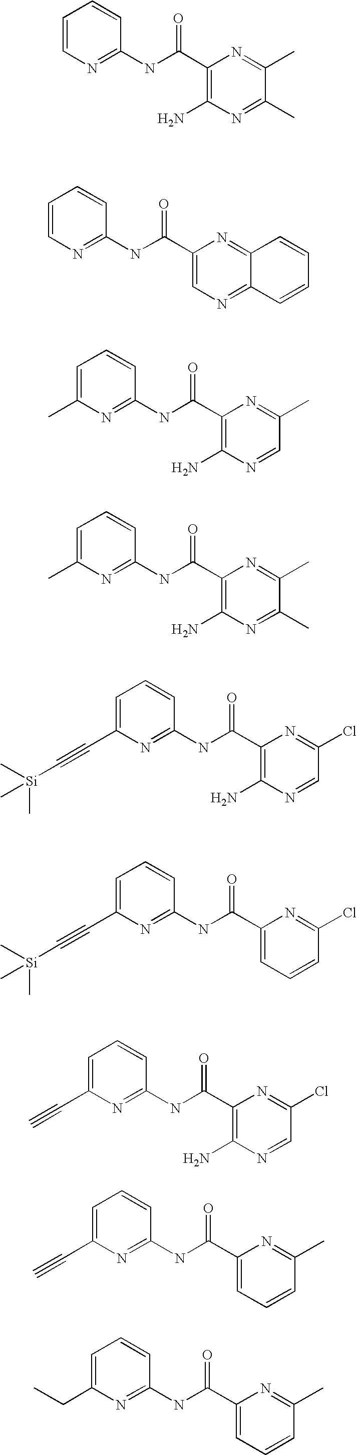 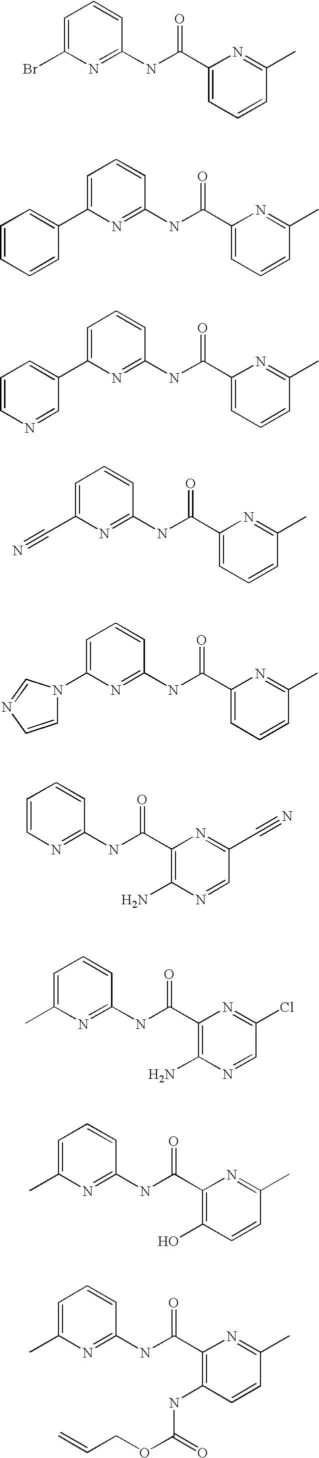 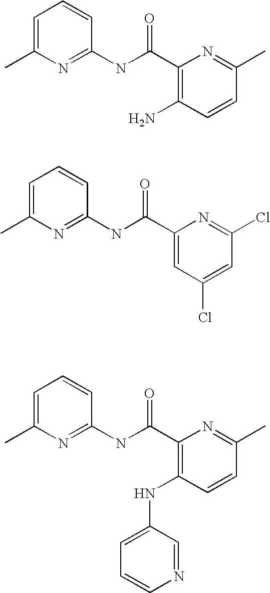
or a pharmaceutically acceptable salt thereof.

16. A pharmaceutical composition comprising a therapeutically effective amount of the compound according to claim 1, 2, 9 or 15, or a pharmaceutically acceptable salt thereof, and a pharmaceutically acceptable carrier.

17. A method of treatment or prevention selected from:
1) treatment or prevention of pain comprising the step of administering a therapeutically effective amount, or a prophylactically effective amount, of the compound according to claim 1 or a pharmaceutically acceptable salt thereof;
2) treatment or prevention of a pain disorder wherein said pain disorder is acute pain, persistent pain, chronic pain, inflammatory pain, or neuropathic pain, comprising the step of administering a therapeutically effective amount, or a prophylactically effective amount, of the compound according to claim 1 or a pharmaceutically acceptable salt thereof;
3) treatment or prevention of anxiety, depression, bipolar disorder, psychosis, drug withdrawal, tobacco withdrawal, memory loss, cognitive impairment, dementia, Alzheimer's disease, schizophrenia or panic comprising the step of administering a therapeutically effective amount, or a prophylactically effective amount, of the compound according to claim 1 or a pharmaceutically acceptable salt thereof;
4) treatment or prevention of Parkinson's disease comprising the step of administering a therapeutically effective amount, or a prophylactically effective amount, of the compound according to claim 1 or a pharmaceutically acceptable salt thereof;
5) treatment or prevention of anxiety disorders comprising the step of administering a therapeutically effective amount, or a prophylactically effective amount, of the compound according to claim 1 or a pharmaceutically acceptable salt thereof;
6) treatment or prevention of epilepsy comprising the step of administering a therapeutically effective amount, or a prophylactically effective amount, of the compound according to claim 1 or a pharmaceutically acceptable salt thereof;
7) treatment or prevention of cognitive dysfunction comprising the step of administering a therapeutically effective amount, or a prophylactically effective amount, of the compound according to claim 1 or a pharmaceutically acceptable salt thereof;
8) treatment or prevention of drug addiction, drug abuse and drug withdrawal comprising the step of administering a therapeutically effective amount, or a prophylactically effective amount, of the compound according to claim 1 or a pharmaceutically acceptable salt thereof;
9) treatment or prevention of circadian rhythm and sleep disorders comprising the step of administering a therapeutically effective amount, or a prophylactically effective amount, of the compound according to claim 1 or a pharmaceutically acceptable salt thereof; and
10) treatment or prevention of obesity comprising the step of administering a therapeutically effective amount, or a prophylactically effective amount, of the compound according to claim 1 or a pharmaceutically acceptable salt thereof.

18. The method of claim 17 wherein said anxiety disorder is panic attack, agoraphobia or specific phobias, obsessive-compulsive disorders, post-traumatic stress disorder, acute stress disorder, generalized anxiety disorder, eating disorder, substance-induced anxiety disorder, or nonspecified anxiety disorder.

19. The method of claim 17 wherein the circadian rhythm and sleep disorders are shift-work induced sleep disorder or jet-lag.
